SEQUENCE LISTING (11 sequences)

SEQ ID NO: 1 - DNA, 48 bp, Artificial Sequence
  Completely synthetic sequence
cctgccacgctccgctcactgacctgggggagtattgcggaggaaggt

SEQ ID NO: 2 - DNA, 48 bp, Artificial Sequence
  Completely synthetic sequence
cctgccacgctccgctcactgacctgggggagtaatgcggagcaaggt

SEQ ID NO: 3 - DNA, 15 bp, Artificial Sequence
  Completely synthetic sequence
gcggagcgtggcagg

SEQ ID NO: 4 - DNA, 11 bp, Artificial Sequence
  Completely synthetic sequence
ccaggtcagtg

SEQ ID NO: 5 - DNA, 44 bp, Artificial Sequence
  Completely synthetic sequence
ttttttttttttcactgacctgggggagtattgcggaggaaggt

SEQ ID NO: 6 - DNA, 44 bp, Artificial Sequence
  Completely synthetic sequence
ttttttttttttcactgacctggggtagtattgcggatgaaggt

SEQ ID NO: 7 - DNA, 11 bp, Artificial Sequence
  Completely synthetic sequence
caggtcagtgt

SEQ ID NO: 8 - DNA, 12 bp, Artificial Sequence
  Completely synthetic sequence
ccaggtcagtgt

SEQ ID NO: 9 - DNA, 13 bp, Artificial Sequence
  Completely synthetic sequence
cccaggtcagtgt

SEQ ID NO: 10 - DNA, 14 bp, Artificial Sequence
  Completely synthetic sequence
ccccaggtcagtgt

SEQ ID NO: 11 - DNA, 15 bp, Artificial Sequence
  Completely synthetic sequence
tccccaggtcagtgt

(19) United States
(12) Patent Application Publication
(10) Pub. No.: US 2006/0068407 A1
(43) Pub. Date: Mar. 30, 2006

(54) Method of immobilizing nucleic acid aptamers

(75) Inventors: Nicholas Rupcich, Oakville (CA); Razvan Nutiu, Hamilton (CA); John D. Brennan, Dundas (CA); Yingfu Li, Dundas (CA)

Correspondence Address: BERESKIN AND PARR, 40 KING STREET WEST, BOX 401, TORONTO, ON, M5H 3Y2, CA

(73) Assignee: McMaster University, Hamilton (CA)

(21) Appl. No.: 11/061,775

(22) Filed: Feb. 22, 2005

Related U.S. Application Data
(60) Provisional application No. 60/545,525, filed on Feb. 19, 2004.

(51) Int. Cl. C12Q 1/68; G01N 1/28

(52) U.S. Cl. 435/6; 427/2.11

(57) ABSTRACT

The present invention relates to methods of immobilizing nucleic acid aptamers within a sol-gel matrix. The aptamer system remains functionally intact when it is immobilized within a protein and membrane-compatible sol-gel derived from polyol silane precursors or sodium silicate.

### RELATED APPLICATIONS

This application claims the benefit of priority under 35 USC §119(e) from U.S. provisional patent application Ser. No. 60/545,525, filed Feb. 19, 2004.

### FIELD OF THE INVENTION

The present invention relates to methods for the immobilization of nucleic acid aptamers, including DNA and RNA aptamers, including DNA or RNA-based catalytic aptamers (sometimes referred to as aptazymes, DNA enzymes, deoxyribozymes or ribozymes), with and without modified nucleotides, to composites prepared by such methods and to the use of these composites, in particular for multianalyte biosensing, metabolite profiling and diagnostics.

### BACKGROUND TO THE INVENTION

Aptamers are single-stranded nucleic acids that are isolated from random-sequence nucleic acid libraries by “in vitro selection”.^{1,2} A large number of DNA or RNA sequences have been isolated that bind a diverse range of targets, including metal ions, small organic compounds, biological cofactors, metabolites, proteins and nucleic acids.^{3,4} The target versatility and the high binding affinity of both DNA and RNA aptamers,^{5,6,7} their properties of precise molecular recognition,^{8,9} along with the simplicity of in vitro selection, make aptamers attractive as molecular receptors and sensing elements.

Since DNA and RNA do not contain any intrinsically fluorescent groups, aptamers can only be made fluorescent through modification with external fluorophores. Three general methods have been described for engineering fluorescent aptamers with real-time signaling capabilities. The first was to modify an aptamer with a fluorophore at a location that undergoes a significant conformational change upon target binding. Such reporters can be either rationally designed based on available tertiary structures¹⁰ or created de novo by in vitro selection using a fluorophore-labeled library.¹¹ The second method was to engineer aptamer beacons^{12,13,14,15,16,17,18,19,20,21} by adopting the same principle used in the design of molecular beacons for real-time detection of nucleic acids.²² The third method is based on fluorescence altering properties of aptamers bound to specific fluorophores.^{23,24,25,26,27} A fourth method is to utilize an aptamer construct that has catalytic function, wherein the DNA or RNA enzyme can be used to act on a fluorogenic substrate to either enhance or quench its fluorescence.²⁸

Recently, the Li group described two methods for obtaining fluorescence signals from DNA aptamers. The first is a simple and general strategy for making solution-based signaling aptamers that function by a coupled structure-switching/fluorescence-dequenching mechanism.²⁹ The approach exploited the unique ability of each and every DNA aptamer to adopt two distinct structures—a DNA duplex with a complementary DNA sequence, and a tertiary complex with a non-nucleic acid target. In their specific designs, the DNA duplex was formed between a fluorophore-labeled DNA aptamer and a small complementary oligonucleotide modified with a quencher. In the absence of the target, the aptamer naturally binds to its complementary sequence, bringing the fluorophore and the quencher into close proximity for high-efficiency fluorescence quenching. When the aptamer target is introduced, the aptamer dissociates from the complementary DNA and forms the aptamer-ligand complex. Since this structure-switching event occurs concomitant with the fluorescence-dequenching event, a strong fluorescence signal is produced. Based on this principle, a structure-switching DNA aptamer that was specific for ATP recognition was developed that could produce a large fluorescent signal upon target introduction.

A second method introduced by the Li group is to use single-stranded DNA molecules with catalytic capabilities, which are isolated from random-sequence DNA libraries by “in vitro selection”. This new class of catalytic biomolecules has the potential of being used as unique molecular tools in a variety of innovative applications. An RNA-cleaving autocatalytic DNA, DEC22-18, is able to uniquely link chemical catalysis with real-time fluorescence signaling capability in the same molecule.²⁸ A trans-acting DNA molecule, DET22-18, was also developed from DEC22-18 that behaves as a true enzyme with a k_{cat} of ˜7 min⁻¹—a rate constant that is the second largest ever reported for a DNA enzyme. It cleaves a chimeric RNA/DNA substrate at the lone RNA linkage surrounded by a closely spaced fluorophore-quencher pair—a unique structure that permits the synchronization of the chemical cleavage with fluorescence signaling. DET22-18 has a stem-loop structure and can be conjugated with DNA aptamers to form allosteric deoxyribozyme biosensors.

To realize the full potential of DNA aptamers, including DNA enzymes for applications such as multianalyte biosensing or metabolite profiling, it is optimal to immobilize the aptamer on a suitable surface, preferably in a microarray format.^{30,31,32} Microarrays can simultaneously measure the expression of thousands of genes, proteins and RNA. Typical methods used to immobilize single stranded DNA are based on covalent, affinity or electrostatic interactions between the DNA strand and a suitable surface. However, unlike standard DNA arrays, which rely on formation of hybridized complexes between immobilized single stranded DNA (ssDNA) and fluorescently labeled complementary analyte DNA, for aptamer-based arrays based on the above structure switching aptamers it is desirable that the immobilized DNA strand be capable of undergoing a significant conformational change that can provide the fluorescent signal, and that the “binding site” for a small molecule remain intact after immobilization.

Recently, the present inventor's group^{33,34} and that of Bright^{35,36,37} have developed microarrays based on pin printing of sol-gel derived silica spots containing biomolecules. Such arrays were used to study enzyme kinetics and inhibition of enzymes entrapped in a pin-printed sol-gel derived medium.³³ Sol-gel entrapment of biomolecules has been proven to have numerous advantages over monolayer-based arraying techniques. These include higher sample loading owing to the three dimensional nature of the sol-gel micro-spot and therefore higher intensity fluorescence levels, as well as improved signal-to-background levels.³⁴ The encapsulated biomolecules remain hydrated and active within the sol-gel material and their stability is increased dramatically over monolayer deposition. However, while sol-gel based materials have been used extensively for the immobilization of active proteins, to date the immobilization of DNA or RNA in such materials has not been reported, although attachment of DNA to the surface of biotinylated sol-gel materials has been described.³⁸

For practical applications, there remains a need for a method of immobilizing nucleic acid aptamers that maintains their ability to act as molecular recognition molecules.

### SUMMARY OF THE INVENTION

The present inventors have developed of a new class of biological microarrays based on the entrapment of an engineered structure-switching DNA aptamer within a pin-printed sol-gel microarray. The fluorescent signaling aptamer system was based on a previously reported construct, and was built using either a tripartite or bipartite construct. The tripartite construct contains three short DNA oligonucleotides: one modified with a fluorophore (denoted FDNA); one labeled with a quencher (QDNA); and the third a DNA aptamer made of a biotinylated adenosine-binding element, an FDNA-binding sequence and a few nucleotides in between. In the bipartite construct the fluorophore is covalently tethered to the aptamer rather than bound to a short complementary DNA strand. In the absence of the target, the DNA molecules were assembled into a tripartite or bipartite duplex structure leading to efficient fluorescence quenching. When the target (ATP) is present, the aptamer prefers the target as its binding partner resulting in the release of QDNA and subsequently a significant increase of fluorescence intensity. It was demonstrated that the tripartite and bipartite aptamer complexes, when bound to steptavidin, remained intact, showed minimal leaching and sustained activity, selectivity and sensitivity to ATP concentration similar to that in solution when entrapped in sodium silicate or diglycerylsilane based glasses. The aptamers could also be immobilized in a pin-printed sol-gel microarray and still retain their characteristic properties, while immobilization of the tripartite aptamers directly onto neutravidin coated slides caused the aptamer to be non-functional. This successful immobilization of DNA aptamers within sol-gel derived microarrays illustrates the power of sol-gel entrapment to concurrently immobilize a range of biological samples, and that metabolomics screening tools can be developed around this technology.

Accordingly, the present invention relates to a method immobilizing nucleic acid aptamers comprising combining at least one nucleic acid aptamer with a sol-gel precursor and treating the at least one aptamer and precursor under conditions for a gel to form.

In an embodiment of the invention, the nucleic acid aptamer is one component of a system. Accordingly, the present invention relates to a method of immobilizing a system comprising at least one nucleic acid aptamer comprising combining the system with a sol-gel precursor and treating the system and precursor under conditions for a gel to form.

The present invention also includes a method of immobilizing nucleic acid aptamers or nucleic acid aptamer systems in silica matrixes comprising:
(i) combining an aqueous solution of a sol-gel precursor with an aqueous solution of the nucleic acid aptamer or nucleic acid aptamer system;
(ii) adjusting the pH of the combination of (i) so that it is in the range of about 4-11;
(iii) shaping the combination into a desired shape;
(iv) allowing the combination to gel; and
(v) aging and partially drying the gel.

Further, the present invention relates to a method for detecting a molecule comprising:
(a) exposing an aptamer, or aptamer system, that specifically binds to the molecule, said aptamer or aptamer system being immobilized in a sol-gel, to a test solution suspected of comprising the molecule; and
(b) detecting a change in one or more characteristics of aptamer or aptamer system.

In another embodiment of the present invention, there is include a method of separating one or more compounds from a mixture comprising:
(a) contacting the mixture with a sol gel comprising an aptamer or aptamer system immobilized therein, said aptamer or aptamer system having selective binding to the one or more compounds, under conditions for the one or more compounds to bind to the aptamer or aptamer system;
(b) treating the sol gel under conditions to remove the mixture from the sol gel; and
(c) optionally isolating the one or more compounds from the sol gel.

As well, the present invention also includes a method of performing chemical reactions comprising:
(a) exposing one or more reactants to a sol gel comprising an aptamer or aptamer system immobilized therein, under conditions for the reaction to proceed to produce one or more reaction products;
(b) removing the one or more reaction products from the sol gel; and optionally isolating the one or more reaction products.

The present invention further relates to sol gels with at least one nucleic acid aptamer, or a system comprising at least one nucleic acid aptamer, immobilized therein.

The present invention also includes kits, biosensors, microarrays, chromatographic and bioaffinity columns comprising the silica matrixes with at least one nucleic acid aptamer, or a system comprising at least one nucleic acid aptamer, immobilized therein, prepared as using a method of the present invention.

Other features and advantages of the present invention will become apparent from the following detailed description. It should be understood; however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1a is a schematic of structure switching activity of an ATP-binding DNA aptamer having a tripartite structure where F=fluorescein tag, Q=Dabcyl tag, and B=biotin. In the absence of analyte the tripartite structure forms with QDNA in close proximity to FDNA, causing extensive quenching. In the presence of analyte, the folding of the aptamer binding site causes displacement of the QDNA, resulting in a dequenching event and hence a large increase in fluorescence intensity.

FIG. 1b is a schematic of the bipartite structure where the fluorophore is covalently tethered to the DNA sequence, thus it is only composed of the aptamer and QDNA. Upon ATP addition the aptamer forms a hairpin loop, which displaces the QDNA, resulting in the fluorescent signal.

FIG. 2 is a graph showing the time dependent changes in fluorescence intensity for the ATP-binding tripartite aptamer in solution and in different sol-gel formulations upon exposure to 1 mM ATP, CTP or UTP.

FIG. 3 contains graphs showing initial reaction rate vs. concentration of ATP (Panel A) and endpoint fluorescence intensity vs ATP concentration (Panel B) following a 1 hour incubation of a tripartite aptamer in various concentrations of ATP. The rates were calculated by measuring the change in fluorescence intensity with time, and 12 time points were used in determining the initial slopes.

FIG. 4 shows (a) pin-printed columns of a tripartite aptamer mutant (M), FDNA (F), blank (B) and ATP tripartite aptamer (A) from left to right onto a streptavidin coated slide (200 μm spots); (b) Same samples printed with sodium silicate onto a GAPS derivatized slide, but at half the concentration and using 100 μm spots.

FIG. 5 shows (a) a sodium silicate array consisting of entrapped fluorescein dextran (row 1), blank (row 2) and a ATP tripartite aptamer (row 3). In duplicate columns are the analytes that the array was exposed to for 3 hours (from 1 to r): buffer, ATP, CTP, UTP, GTP; (b) a bar graph illustrating the selectivity of an entrapped active ATP tripartite aptamer for 1 mM ATP. The 10-fold enhancement was only witnessed for the aptamer sample exposed to 1 mM ATP, while the remainder of the spots remained relatively unchanged. The enhancement is measured and normalized relative to the buffer incubated sample.

FIG. 6 is a plot of tripartite aptamer/mutant ratio with respect to ATP concentration.

FIG. 7 is a Stern-Volmer plot of the quenching of fluorescence for the entrapped bipartite aptamer by iodide at different time points following exposure to the anionic quencher: (▪) solution; (●) 20 min incubation time; (◯) 60 min incubation time; (▾) 24 h incubation time.

FIG. 8 is a graph showing the Fluorescence signaling ability of the ATP-binding aptamer upon exposure to 2 mM ATP when entrapped in five different sol-gel materials: SS (●), SS+0.1% APTES (□), DGS (▪), DGS+0.1% APTES (⋄) and TEOS (▴). Also shown are the responses for the tripartite (Δ) and bipartite (◯) aptamer in solution.

FIG. 9 is a graph showing the fluorescence signaling ability of the bipartite aptamer bearing QDNA strands of varying length. The change in fluorescence signal upon exposure to 0.5 mM ATP is shown on the graph for each system: (●) Q10DNA; (◯) Q11DNA; (▾) Q12DNA; (Δ) Q13DNA (▪) Q15DNA.

FIG. 10 is a graph showing the comparison of selectivity of the tripartite aptamer and an inactive mutant version of the aptamer. All analyses were done using 0.5 mM of the specified analyte, which correlates to the K_{d} of the aptamer for ATP: (●) Aptamer+ATP; (◯) blank; (▾) Mutant+ATP; (Δ) Aptamer+CTP; (▪) Aptamer+UTP; (□) Aptamer+GTP; (▴) Fluorescein dextran.

FIG. 11 shows (a) Temporal response curves of the tripartite aptamer to varying concentrations of ATP when entrapped in SS derived materials, (b) response curve showing fluorescence endpoint vs. [ATP] following incubation for 1 h, (c) response curve showing initial slope vs. [ATP]. Note the shape of curves of both (b) and (c) follow the response of the aptamer in solution. The rates were calculated by measuring the change in fluorescence with time, and 12 time points were used in determining the initial slopes: (●) Buffer; (◯) 0.01 mM ATP; (▾) 0.05 mM ATP; (Δ) 0.1 mM ATP; (▪) 0.5 mM ATP; (□) 1 mM ATP; (♦) 2 mM ATP; (⋄) 3 mM ATP; (▴) 4 mM ATP; (Δ) 5 mM ATP.

### DETAILED DESCRIPTION OF THE INVENTION

(I) Method of Entrapping Nucleic Acid Aptamers

The work described herein provides the first example of aptamer entrapment within sol-gel derived materials, including microarrays, and establishes sol-gel materials as a viable alternative to direct immobilization via avidin-biotin interactions for signaling aptamer microarrays. Aptamers can be selected for virtually any biological target from small metabolites to large proteins and in conjunction with the microarray format, provide a tool for rapid diagnostic analysis of complex biological samples.

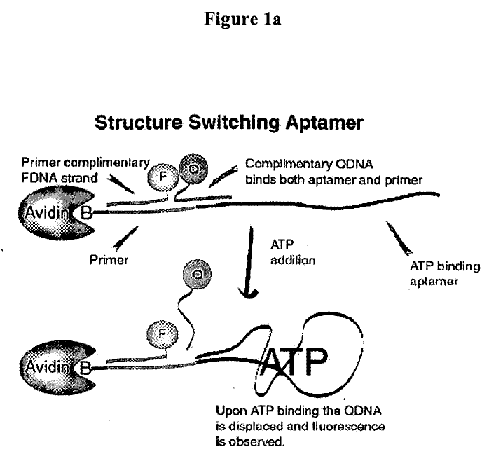

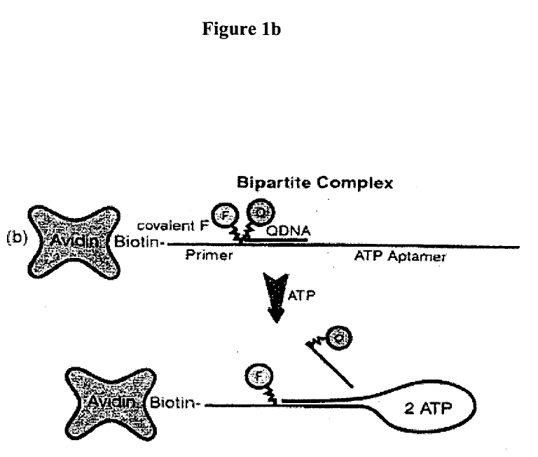

The present invention describes the successful entrapment of two forms of structure-switching DNA aptamers into biocompatible sol-gel derived materials. These include a tripartite construct (FIG. 1a) and a bipartite construct wherein the fluorophore group is covalently tethered to the aptamer rather than bound to a short complementary DNA strand (FIG. 1b). This construct should eliminate the possibility of false signals arising from FDNA displacement upon entrapment and potentially increase the reversibility of the complex. Overall, the data obtained and described herein below indicate that the sol-gel entrapment method will be amenable to the development of devices that require immobilized DNA aptamers.

Accordingly, the present invention relates to a method of immobilizing nucleic acid aptamers comprising combining at least one nucleic acid aptamer with a sol-gel precursor and treating the at least one apatmer and precursor under conditions for a gel to form.

In an embodiment of the invention, the nucleic acid aptamer is one component of a system. Accordingly, the present invention relates to a method of immobilizing a system comprising at least one nucleic acid aptamer comprising combining the system with a sol-gel precursor and treating the system and precursor under conditions for a gel to form.

The term “aptamer” as used herein refers to both deoxyribonucleic and ribonucleic acid molecules (i.e. DNA and RNA) which are artificial nucleic acid ligands that can be generated by in vitro selection against a diverse range of targets, including, for example, metal ions, small organic molecules, biological cofactors, metabolites, proteins and nucleic acids. This includes natural RNA aptamers denoted riboswitches.^{39,40,41,42,43,44,45,46,47} The aptamer may also be modified to increase stability, for example, by modification of the 2′-positions of pyrimidine nucleotides with amino/fluoro groups^{48,49}. The aptamer may also be labelled, for example with a fluorescent label, or it may be part of a functional aptamer system. The term “aptamer system” as used herein refers to aptamers that are used in combination with other functional components which are either covalently linked to the aptamer, joined via hybidization or other electrostatic interactions to the aptamer or are simply mixed together in solution with the aptamer. Examples of aptamer systems include the structure switching DNA aptamer described by Li²⁹, aptamer beacons,¹²⁻²⁰ fluorophore-binding aptamers,²³⁻²⁷ and aptamers used in combination with other reporter molecules, for example, antibodies. Examples of structure-switching aptamers are described in inventor Li's co-pending patent applications: entitled “Signaling aptamer complex”, PCT/CA2003/00086 filed on Jan. 22, 2003 and “Aptamer selection method”, PCT/CA2004/000482 filed on Mar. 31, 2004, the contents of all of which are incorporated herein by reference. The aptamer may also be a catalytic aptamer that is capable of coupling catalysis with fluorescence signalling. Examples of such catalytic aptamers are described in inventor Li's co-pending patent applications entitled “DNA Enzymes” PCT/CA2003/00198 filed on Feb. 14, 2003, and “Metal ion specific and pH dependent DNA enzymes”, PCT/CA2004/000330 filed on Mar. 7, 2004, the contents of all of which are incorporated herein by reference.

In an embodiment of the present invention, the aptamer is part of a functional system, in particular a structure switching aptamer system comprising:
(a) a DNA or RNA that has been modified by addition of a fluorophore (denoted FDNA or FRNA);
(b) a DNA or RNA that has been modified by addition of a molecule that quenches fluorescence of the fluorophore (denoted QDNA or QRNA); and
(c) a DNA or RNA aptamer which comprises a nucleotide sequence that specifically binds to an analyte of interest, a nucleotide sequence that binds to the FDNA or FRNA and a nucleotide sequence that binds to the QDNA or QRNA.
   In embodiments of the invention, the nucleotide sequence that specifically binds to an analyte of interest is biotinylated. The three DNA or RNA molecules are assembled, for example by hydridization, into a tripartite structure.

In a further embodiment of the invention, the structure switching aptamer system comprises:
(a) a DNA or RNA that has been modified by addition of a molecule that quenches fluorescence of a fluorophore (denoted QDNA or QRNA); and
(b) a DNA or RNA aptamer which comprises a nucleotide sequence that specifically binds to an analyte of interest, a nucleotide sequence that has been modified by addition of the fluorophore and a nucleotide sequence that binds to the QDNA or QRNA.

In the above embodiment of the invention, the nucleotide sequence that specifically binds to an analyte of interest may also be biotinylated. The two DNA or RNA molecules are assembled, for example by hydridization, into a bipartite structure.

The term “fluorophore” as used herein means a molecule comprising fluorescent properties that can be detected by, for example, using confocal microscopy. The fluorophore may be any suitable molecule that is compatible for use with DNA or RNA aptamers, for example, fluorescein, Alexafluor dyes, rhodamine-based dyes, and any other fluorescent dye that can be excited using light in the ultraviolet, visible or near infrared part of the spectrum. The selection of quenching molecules will depend on the identity of the fluorophore and could easily be determined by a person skilled in the art. Typical examples include Dabcyl, nanogold, QSY, and the “Blackhole” quenchers available from Molecular Probes.

The “analyte of interest” may be any compound for which a selective DNA or RNA aptamer may be generated. The analyte may be, for example, metal ions, small organic molecules, biological cofactors, metabolites, proteins and nucleic acids. In an embodiment of the invention, the analyte of interest is adenosine triphosphate (ATP).

In embodiments of the present invention, the QDNA or QRNA comprises from 10 to 15 nucleotides. In further embodiments of the invention, FDNA or QRNA comprises from 11 to 12 nulceotides.

In a specific embodiment of the present invention, the aptamer system comprises [SEQ ID NO: 1], [SEQ ID NO: 3] and [SEQ ID NO: 4]. In a further specific embodiment of the present invention, the aptamer system comprises [SEQ ID NO: 5] and a nucleic acid sequence selected from the group consisting of [SEQ ID NO: 7], [SEQ ID NO: 8], [SEQ ID NO: 9], [SEQ ID NO: 10] and [SEQ ID NO: 11]. In a still further specific embodiment of the present invention, the aptamer system comprises [SEQ ID NO: 5] and a nucleic acid sequence selected from the group consisting of [SEQ ID NO: 8] and [SEQ ID NO: 9].

In a further embodiment of the invention, the aptamer system comprises a signaling DNA enzyme construct comprising:
(a) an enzymatic DNA sequence; and
(b) a DNA sequence having a ribonucleotide linkage flanked by a fluorophore modified oligonucleotide and a quencher modified oligonucleotide in sufficient proximity to each other whereby, in the absence of catalysis, fluorescence from the fluorophore is quenched by the quencher.

In a further embodiment, the signaling DNA sequence construct further comprises an aptamer sequence conjugated to the enzymatic DNA sequence

In an embodiment of the present invention, the aptamer or aptamer system is bound (either covalently or electrostatically or by affinity interactions) to another molecule that inhibits leaching of the aptamer or aptamer system from the sol-gel matrix. Examples of such molecules include, for example, streptavidin, which can be used to bind biotinylated nucleic acids.

As used herein, the term “immobilized” means that the aptamer or aptamer system is physically, electrostatically or otherwise confined within the nanometer-scale pores of the biomolecule-compatible matrix. In an embodiment of the invention, the aptamer or aptamer system does not associate with the matrix, and thus is free to rotate within the solvent-filled pores. In a further embodiment of the invention, the aptamer or aptamer system is optionally further immobilized through electrostatic, hydrogen-bonding, bioaffinity, covalent interactions or combinations thereof, between the aptamer or aptamer system and the matrix. In a specific embodiment, the immobilization is by physical immobilization within nanoscale pores.

The sol-gel precursor may be any such compound that leads to the formation of sol-gels under conditions that are compatible with aptamers and aptamer systems. By “compatible” it means that the conditions do not lead to denaturation and therefore loss of activity of the aptamer or aptamer system. In an embodiment of the invention, the sol-gel precursors are biomolecule compatible. By “biomolecule-compatible” it is meant that the conditions for forming the sol-gel and the resulting silica matrix either stabilizes nucleic acids, proteins, membranes and/or other biomolecules against denaturation or do not facilitate denaturation. The term “biomolecule” as used herein means any of a wide variety of nucleic acids including DNA and RNA, proteins, enzymes, organic and inorganic chemicals, other sensitive biopolymers and complex systems including whole or fragments of plant, animal and microbial cells that may be entrapped in the matrix.

In the invention, the biomolecule-compatible matrix (i.e. the sol-gel) is prepared using biomolecule-compatible techniques, i.e. the preparation involves biomolecule-compatible precursors and reaction conditions that are biomolecule-compatible. In a further embodiment of the invention, the biomolecule-compatible sol gel is prepared from a sodium silicate precursor solution. In still further embodiments, the sol gel is prepared from organic polyol silane precursors. Examples of the preparation of biomolecule-compatible sol gels from organic polyol silane precursors are described in inventor Brennan's co-pending patent applications entitled “Polyol-Modified Silanes as Precursors for Silica”, PCT patent application publication number WO03/102001, filed on Jun. 2, 2003 and corresponding U.S. patent application publication number US2004-0034203, filed on Jun. 2, 2003; and “Methods and Compounds for Controlling the Morphology and Shrinkage of Silica Derived from Polyol-Modified Silanes”, PCT patent application publication number WO 04/018360, filed Aug. 25, 2003, and corresponding U.S. patent application publication number US2004-0249082, filed on Aug. 25, 2003, the contents of all of which are incorporated herein by reference. In specific embodiments of the invention, the organic polyol silane precursor is prepared by reacting an alkoxysilane, for example tetraethoxysilane (TEOS) or tetramethoxysilane (TMOS), with an organic polyol. In an embodiment, the organic polyol is selected from sugar alcohols, sugar acids, saccharides, oligosaccharides and polysaccharides. Simple saccharides are also known as carbohydrates or sugars. Carbohydrates may be defined as polyhydroxy aldehydes or ketones or substances that hydroylze to yield such compounds. The organic polyol may be a monosaccharide, the simplest of the sugars, or a carbohydrate. The monosaccharide may be any aldo- or keto-triose, pentose, hexose or heptose, in either the open-chained or cyclic form. Examples of monosaccharides that may be used in the present invention include one or more of allose, altrose, glucose, mannose, gulose, idose, galactose, talose, ribose, arabinose, xylose, lyxose, threose, erythrose, glyceraldehydes, sorbose, fructose, dextrose, levulose and sorbitol. The organic polyol may also be a disaccharide, for example, one or more of, sucrose, maltose, cellobiose and lactose. Polyols also include polysaccharides, for example one or more of dextran, (500-50,000 MW), amylose and pectin. In embodiments of the invention the organic polyol is selected from one or more of glycerol, sorbitol, maltose, trehelose, glucose, sucrose, amylose, pectin, lactose, fructose, dextrose and dextran and the like. In embodiments of the present invention, the organic polyol is selected from glycerol, sorbitol, maltose and dextran. Some representative examples of the resulting polyol silane precursors suitable for use in the methods of the invention include one or more of diglycerylsilane (DGS), monosorbitylsilane (MSS), monomaltosylsilane (MMS), dimaltosylsilane (DMS) and a dextran-based silane (DS). In embodiments, the polyol silane precursor is selected from one or more of DGS and MSS, specifically DGS.

In further embodiments of the invention, the biomolecule-compatible matrix precursor is selected from one or more of functionalized or non-functionalized alkoxysilanes, polyolsilanes or sugarsilanes; functionalized or non-functionalized bis-silanes of the structure (RO)₃Si—R′—Si(OR)₃, where R may be ethoxy, methoxy or other alkoxy, polyol or sugar groups and R′ is a functional group containing at least one carbon (examples may include hydrocarbons, polyethers, amino acids or any other non-hydrolyzable group that can form a covalent bond to silicon); functionalized or non-functionalized chlorosilanes; and sugar, polymer, polyol or amino acid substituted silicates.

In yet another embodiment of the present invention, the biomolecule compatible matrix further comprises an effective amount of one or more additives. In embodiments of the invention the additives are present in an amount effective to enhance the mechanical, chemical and/or thermal stability of the matrix and/or assembly components, or to aid in printability of the composition in a microarray format. In an embodiment, the mechanical, chemical and/or thermal stability is imparted by a combination of precursors and/or additives, and by choice of aging and drying methods. Such techniques are known to those skilled in the art. In further embodiments of the invention, the additives are selected from one or more of humectants and other protein stabilizing agents (for e.g. osmolytes). Such additives include, for example, one or more of organic polyols, hydrophilic, hydrophobic, neutral or charged organic polymers, block or random co-polymers, polyelectrolytes, sugars (natural or synthetic), and amino acids (natural and synthetic). In embodiments of the invention, the one or more additives are selected from one or more of glycerol, sorbitol, sarcosine and polyethylene glycol (PEG). In further embodiments, the additive is glycerol.

In a particular embodiment of the invention the biocompatible matrix is a sol-gel prepared from, for example, a silicon alkoxide, alkylated metal alkoxide or otherwise functionalized metal alkoxide or a corresponding metal chloride, silazane, polyglycerylsilicate, diglycerylsilane or other silicate precursor, optionally in combination with additives selected from one or more of any available organic polymer, polyelectrolyte, sugar (natural or synthetic) or amino acids (natural and non natural). The preparation of sodium silicate solutions for use as a sol-gel precursor is known in the art.³⁸

The aptamer or aptamer system may be combined with a biomolecule-compatible, sol-gel precursor solution under conditions for a gel to form. By “gel” it is meant a solution or “sol” that has lost flow. The sols lose flow due to the hydrolysis and polycondensation of the precursor. The hydrolysis and condensation of the polyol silane and sodium silicate precursors may suitably be carried out in aqueous solution. Suitably, a solution, for example a homogeneous solution, of precursor, in acidified water is used, or in the case of DGS a solution of the precursor in water or buffer at neutral pH. Sonication may be used in order to obtain a homogeneous solution. By “homogeneous” it is meant having an essentially uniform composition or structure. Conditions for the formation of a gel comprise adjusting the pH of the aqueous solution of precursor so that formation of a gel occurs. Suitably, the pH may be in the range of about 4-11. The pH may be adjusted, for example, by the addition of suitable buffer solutions or resins, for example Dowex resin. As the solutions lose flow, they can be formed, cast, moulded, shaped, spun, pin-printed as microarrays or drawn into desired shapes. Examples of such shapes include, but are not limited to, films, spots, fibres, monoliths, pellets, granules, tablets, rods or bulk. The solutions may also be placed into multi-well plates for high-throughput screening applications, or printed as microarrays for multianalyte sensing or screening. Accordingly, in an embodiment of the present invention, the method of immobilizing nucleic acid aptamers or nucleic acid aptamer systems in silica matrixes comprises:
(i) combining an aqueous solution of a sol-gel precursor with an aqueous solution of the nucleic acid aptamer or nucleic acid aptamer system;
(ii) adjusting the pH of the combination of (i) so that it is in the range of about 4-11;
(iii) shaping the combination into a desired shape;
(iv) allowing the combination to gel; and
(v) aging and partially drying the gel.

In an embodiment of the invention, the nucleic acid aptamer or nucleic acid aptamer system is combined with the sol-gel precursor in a ratio of about 1:1.

A person skilled in the art would appreciate that the conditions may need to be adjusted depending on the identity of the sol-gel precursor and the aptamer or aptamer system and could do so without undue experimentation in light of the present disclosure and the examples provided herein.

Once the gel has been formed and shaped it may be aged over a period of time under select conditions to lock the conformation of the gel, its pores, matrixes and interconnecting channels into fixed positions and permit long term storage. In embodiments of the invention, the gels are aged in buffer or in a solution comprising an effective amount of a humectant, for example glycerol (suitably about 5-50% (v/v) of glycerol in water or buffer solution, suitably 25% (v/v) of glycerol in water or buffer solution).

The present invention further relates to sol-gels with one or more aptamers or aptamer systems immobilized therein and prepared using the method as described hereinabove.

The term “a” as used herein, unless otherwise indicated, also denotes “one or more”.

(II) Uses

The immobilization of aptamers is important in several technologies including use as analytical reagents, for example in the development of biosensors, microarrays and bioaffinity columns, and as enzymes or catalysts for chemical reactions. The sol-gels prepared using the method described in the previous section can be used for any of these applications.

In applications as analytical tools, immobilization of aptamers in sol gels has advantages in, for example biosensors, and in analytic separation techniques. In these applications, it is the aptamer's ability to specifically recognize and bind to a wide range of chemical entities, that provides its usefulness. The sol gel comprising a nucleic acid aptamer for use as analytical tools may be conveniently prepared as columns or as microarrays.

Accordingly, also included within the scope of the present invention is a method for detecting a molecule comprising:
(a) exposing an aptamer, or aptamer system, that specifically binds to the molecule, said aptamer or aptamer system being immobilized in a sol-gel, to a test solution suspected of comprising the molecule; and
(b) detecting a change in one or more characteristics of aptamer or aptamer system.
   In embodiments of the invention, the sol-gel is prepared using a method described herein. In further embodiments of the invention, a change in the one or more characteristics of the aptamer or aptamer system in the presence of the molecule compared to one or more controls indicates that the molecule was present in the solution.

By “control” is meant either positive or negative controls, for example repeating the same method, under the same conditions but in the absence (negative control) or presence (positive control) of the molecule.

The molecule can be any compound which one wishes to detect including, but not limited to, proteins (including antibodies), peptides, nucleic acids, fragments of proteins, peptides, carbohydrates, organic compounds, inorganic compounds and natural products. The test solution suspected of comprising the molecule may be, for example, a reaction mixture, library extracts, bodily fluids and other samples that one wishes to test for the presence of the molecule. The molecule may be in liquid or gaseous form.

Such assay systems are most conveniently “miniaturized” through any acceptable method of miniaturization, including but not limited to multi-well plates, such as 24, 48, 96 or 384-wells per plate, microfluidic chips, microarrays or slides. The assay may be reduced in size to be conducted on a microfluidic-chip support, advantageously involving smaller amounts of reagents and other materials. Any miniaturization of the process which is conducive to high-throughput screening is within the scope of the invention.

In an embodiment of the invention, the sol-gel entrapped aptamers and aptamer systems are formed into microarrays. Microarrays may be formed by pin-printing the solution comprising the aptamer or aptamer system and the sol-gel precursors onto a suitable surface in array format before the solution gels. The solutions are then allowed to gel and dry on the surface. Suitable methods for forming sol-gel microarrays are known in the art (see, for example, inventor Brennan's co-pending PCT Patent Application publication number WO 2004/039487 and U.S. regular application Ser. No. 10/815,727 entitled “Multicomponent Protein Microarrays”, both filed on Nov. 3, 2003). The present invention provides the first example of the successful (i.e. maintaining of aptamer function) immobilzation of aptamers in sol gel-based microarrays.

The “one or more characteristics of the aptamer or aptamer system” that may be used to detect molecules include, but are not limited to, changes in structure orientation as evidenced by changes in the fluorescent signal of a fluorescent indicator molecule attached (either covalently or electrostatically) to the aptamer or aptamer system, or binding to fluorescently labelled antibodies. Fluorescence is only one of many means of detecting change in one or more characteristics of the aptamer or aptamer system. Because of the light-transmission capabilities of the matrixes of the present invention, UV, IR and visible light optical spectroscopy, as well as luminescence, absorption, emission, excitation and reflection techniques are all suitable for detecting changes in the characteristics of the entrapped aptamer or aptamer system.

In an embodiment of the invention, the aptamer system is a structure-switching nucleic acid aptamer. In these systems, binding of the molecular target for the aptamer results in an increase in fluorescence intensity due to release of a fluorescence quenching molecule that was previously bound to the aptamer in close proximity to a fluorescent indicator (also bound, covalently or electrostatically, to the aptamer). In this example, the presence of the target molecule in the test solution results in an increased fluorescence signal for the aptamer system immobilized in the sol gel.

In further embodiments of the invention, the method for the detection of molecules is performed in a quantitative fashion so that the amount of molecule in the test solution can be determined.

In yet another embodiment of the present invention, the immobilized aptamers and aptamer systems of the present invention can be used to separate and purify compounds. Accordingly, the present invention further relates to a method of separating one or more compounds from a mixture comprising:
(a) contacting the mixture with a sol gel comprising an aptamer or aptamer system immobilized therein, said aptamer or aptamer system having selective binding to the one or more compounds, under conditions for the one or more compounds to bind to the aptamer or aptamer system;
(b) treating the sol gel under conditions to remove the mixture from the sol gel; and
(c) optionally isolating the one or more compounds from the sol gel.

In embodiments of the invention, the sol gel comprising an aptamer or aptamer system may be used to separate one or more unwanted compounds from a mixture, accordingly, in this embodiment, the unwanted compound(s) will bind to the aptamer or aptamer system immobilized in the sol-gel and the desired compound(s) may be isolated from the material removed from the sol-gel.

By “selective binding” it is meant that the aptamer or aptamer system preferentially binds the compound(s) to be retained within the sol-gel over the compound(s) to be removed from the sol-gel to provide efficient and useful amounts of separation of unwanted compound(s) from wanted compound(s).

In the method of separating one or more compounds from a mixture using the sol gels of the present invention, it is convenient for the sol gel to be formed into a column, for example a chromatographic column. A person skilled in the art would appreciate that the “conditions to allow the one or more compounds to bind to the aptamer or aptamer system” and “conditions to remove the mixture from the sol gel” would vary depending on the compound(s) and aptamer or aptamer system, and could be determined using standard techniques known to those skilled in the art.

Compounds that may be separated using the method of the invention include any compound for which a specific recognition aptamer can be prepared and includes, but is not limited to, proteins (including antibodies), peptides, nucleic acids, fragments of proteins, peptides, carbohydrates, organic compounds, inorganic compounds and natural products, including all isomers thereof. The mixture may be, for example, a reaction mixture, library extracts, bodily fluids, racemic mixtures and other mixtures from which one wishes to isolate the compound(s).

In yet another embodiment of the present invention, the sol gels of the present invention comprising an aptamer or aptamer system may be used to perform solid phase reactions. Nucleic acid aptamers have been used as enzymes or catalysts in chemical reactions, accordingly, such reactions may be performed within the matrixes of the sol gels of the present invention avoiding the need to separate the enzyme from the reaction mixture after the reaction is complete. Therefore, the present invention further relates to a method of performing chemical reactions comprising:
(a) exposing one or more reactants to a sol gel comprising an aptamer or aptamer system immobilized therein, under conditions for the reaction to proceed to produce one or more reaction products;
(b) removing the one or more reaction products from the sol gel; and
(c) optionally isolating the one or more reaction products.

The present invention also includes kits, biosensors, microarrays, chromatographic and bioaffinity columns comprising the silica matrixes having an aptamer or aptamer system according to the present invention immobilized therein. The kits of the present application comprise, in different combinations, sol gel precursors, reagents for use with the precursors, the aptamer or aptamer system, signal detection and processing instruments, databases and analysis and database management software above. Alternatively the kits may comprise sol gels with an apatemr or aptamer system immobilized therein and reagents for use therewith.

Yet another aspect of the present invention provides a method of conducting a target discovery business comprising:
(a) providing one or more assay systems for detecting or separating compounds, said assay systems using a method of the invention;
(b) (optionally) conducting therapeutic profiling of the compounds identified in step (a) for efficacy and toxicity in animals; and
(c) licensing, to a third party, the rights for further drug development and/or sales of compounds identified in step (a), or analogs thereof.

The following non-limiting examples are illustrative of the present invention:

### EXAMPLES

### Example 1

### Study on the Development of the Structure Switching Aptamer Microarrays with the Tripartite Aptamer Complex based on the Sol-Gel Pin Printing Method

Materials and Methods

Chemicals. Dowex 50×8-100 cation exchange resin, Tris buffer, adenosine triphosphate (trisodium salt, ATP) and glycerol were obtained from Sigma (St. Louis, Mo.). γ-aminopropylsilane (GAPS) derivatized glass microscope slides were purchased from Corning (Corning, N.Y.) and neutravidin coated slides were obtained from Xenopore (Hawthorne, N.J.). Sodium silicate (SS, technical grade, 9% Na₂O, 29% silica, 62% water) and 3-aminopropyltriethoxysilane (APTES) were purchased from Fisher Scientific (Pittsburgh, Pa.). Fluorescein dextran (FD, 70,000 MW) was obtained from Molecular Probes (Eugene, Oreg.). Diglycerylsilane (DGS) was prepared as described elsewhere.⁵⁰ Water was purified with a Milli-Q Synthesis A10 water purification system. All other chemicals and solvents used were of analytical grade.

Procedures

Preparation of DNA Aptamers: Structure-switching DNA aptamers were prepared using protocols that are described elsewhere.²⁹ The specific DNA sequences used in this work were as follows:

Aptamer:

| | |
|---|---|
| 5′-Biotin-CCTGCCACGCTCCGCTCACTGACCTGGGGGAGTATTGCGGAGGAAGGT-3′ | [SEQ ID NO:1] |

Mutant:

| | |
|---|---|
| 5′-Biotin-CCTGCCACGCTCCGCTCACTGACCTGGGGGAGTAATGCGGAGCAAGGT-3′ | [SEQ ID NO:2] |
| FDNA: 5′-Fluorescein-C₆-GCGGAGCGTGGCAGG-3′ | [SEQ ID NO:3] |
| QDNA: 5′-CCAGGTCAGTG-C₆-Dabcyl-3′ | [SEQ ID NO:4] |

The lighter colored text refers to the FDNA binding area, the italicized region designates the QDNA binding site and the mutations present in the mutant DNA are underlined.

Preparation of Sol-Gel Entrapped DNA Aptamer Complex: Stock solutions of each DNA form were prepared at a concentration of 10 μM in a 20 mM Tris buffer at pH 8.3 containing 100 mM NaCl and 5 mM MgCl₂. The tripartite complex was prepared by combining 34 μL of either the aptamer or mutant with 17 μL of FDNA and 51 μL of QDNA. This 1:2:3 ratio of FDNA:aptamer:QDNA ensured that the majority of the FDNA would be annealed to the aptamer while the excess QDNA ensured proper quenching and low background fluorescence. Samples mixed with sol-gel precursors had an additional 50 μL of streptavidin (1 mg.ml⁻¹) added to them to produce an aptamer:streptavidin complex which helped to prevent leaching of the aptamer from the sol-gel matrix. DNA samples that were printed directly onto streptavidin coated slides were prepared at twice the concentration of DNA samples used for entrapment in sol-gel derived silica, and included glycerol at a level of 40% v/v to improve the printability of the aqueous samples.

A sodium silicate solution (SS) was prepared by diluting 2.9 g of sodium silicate in 10 mL of ddH₂O and immediately adding 5 g of the Dowex resin. The mixture was stirred for 30 seconds and then vacuum filtered through a Buckner funnel. The filtrate was then further filtered through a 0.45 μM membrane syringe filter to remove any particulates in the solution. Spotting solutions were formed by combining the precursor solution and the buffered DNA sample solutions in a 1:1 (v/v) ratio in the well of a 96-well plate. Final reagent concentrations in the spotting solutions were as follows: 1.1 μM aptamer, 0.55 μM FDNA, 1.65 μM QDNA and 2.25 μM streptavidin.

Optimization of Sol-Gel Compositions: Optimization of sol-gel materials and surface treatments for the preparation of sol-gel derived microarrays was described previously,³⁴ and all sol-gel based materials used in this work were based on printable formulations. Silicates were formed using either sodium silicate or diglycerylsilane (DGS) as the starting precursor, with or without 0.1% v/v APTES added. Optimal sol-gel compositions were determined by examining the ability of the aptamer to bind to ATP and produce a fluorescence signal when entrapped into a sol-gel derived disk present in a 96 well plate. In each case the total sample volume was 40 μL and samples were aged in air for 6 hours. Following aging, the samples were incubated with 100 μL of buffer for at least 2 hours. Following buffer incubation, the buffer was removed from above the sample and replaced with 100 μL of a solution containing varying concentrations of ATP. Emission intensity was monitored at a wavelength of 525 nm (λₑₓ=490 nm) using a TECAN Safire absorbance/fluorescence platereader operated in fluorescence mode, using the bottom read format.

Microarray Pin-Printing and Imaging. A Virtek Chipwriter Pro (Virtek Engineering Sciences Inc., Toronto, ON) robotic pinspotter equipped with a SMP 3 stealth microspotting pin (250 nL uptake, 0.6 nL delivery, Telechem Inc., Sunnyvale, Calif.) was used to print samples onto both GAPS derivatized glass microscope slides (sol-gel arrays) or streptavidin coated slides (directly printed monolayer arrays) from 96-well plates using a printhead speed of 16 mm.s⁻¹. Printing was done at room temperature with a relative humidity of approximately 50-70%. Fluorescence images of the microarrays were taken with a Carl Zeiss 510 confocal microscope using a multi-line argon ion laser source for excitation of fluorescein (488 nm).

ATP Binding Assays on Arrays: Sol-gel derived microarrays were aged in air for 18-24 hours following preparation and then incubated with buffer for 3 hours prior to exposure to 15 μL of a solutions containing varying levels of ATP to the top of the each array. ATP exposure lasted for 3 hours, after which the solution was removed by gently blowing air over the slide (Note: it was optimal to remove the sample spot as the presence of the aqueous bead on the microarray surface impeded imaging of the array). Streptavidin coated slides printed with aqueous samples were incubated for 48 hours to allow the biotinylated DNA to completely bind to the streptavidin, and then treated identically to the sol-gel derived samples. Following sample incubation with the ATP and the substrate removal, all samples were imaged using confocal fluorescence microscopy, as described elsewhere.³⁴

Preparation of DNA Structure Switching Aptamer

The preparation and characterization of the ATP-binding structure-switching aptamer has been described elsewhere.²⁹ Briefly, the aptamer consists of three interacting parts: the ATP-binding DNA aptamer strand; the FDNA strand which contains a fluorescein label and compliments a primer extension of the aptamer strand; and the QDNA strand which is labeled with the quenching molecule Dabcyl and is complimentary to a part of the same primer extension and part of the aptamer sequence, as shown in FIG. 1a. Upon binding of two ATP molecules the single strand of the aptamer undergoes a structural switch to form a hairpin-type loop,²⁹ resulting in displacement of the QDNA strand and a concurrent decrease in fluorescence quenching of the adjacent fluorescein on the FDNA strand, producing a large increase in fluorescence intensity.

Properties of Entrapped Aptamer

It is desirable for proper performance of the entrapped aptamer that: 1) the aptamer does not leach from the matrix; 2) the tripartite structure remain fully associated upon entrapment; 3) the aptamer be accessible to externally added ATP; 4) the aptamer be able to undergo the structural switch upon binding ATP; and 5) the QDNA be able to move sufficiently far from the FDNA to produce a large fluorescence intensity increase. In addition, entrapment should provide the ability to regenerate the aptamer system by adding QDNA, allowing the immobilized aptamer to be reused for multiple assays. Leaching results were performed in each of the four sol-gel derived samples (DGS, DGS with 0.1% APTES, SS and SS with 0.1% APTES) for the biotinylated aptamer both in the absence of streptavidin and after formation of an aptamer-streptavidin complex. APTES was added to provide a cationic functionality within the glass that could potentially aid in the retention of the DNA aptamer through the electrostatic interactions, and also to balance the anionic surface charge on the silica so as to minimize electrostatic repulsion of ATP from the matrix.⁵¹ The results indicated that over 50% of the entrapped aptamer leached from DGS and SS glasses when no streptavidin was present. Given that both the DNA backbone and the silica are anionic, and that the aptamer is relatively small (48 nt) it is not surprising that DNA would partition out of the silica matrix. Somewhat less leaching was observed in APTES doped glasses (ca 20%), likely owing to electrostatic interactions between the anionic phosphate groups in the aptamer backbone and the cationic amine groups of APTES. However, as described below, the presence of APTES prevented structure-switching, and thus could not be used in the formation of the silica matrix. The aptamer-streptavidin complex showed only minor leaching (<10%) upon repeated washing in SS and DGS glasses, with the leached aptamer likely originating from the surface of the silica material. The results are consistent with the presence of streptavidin providing a large molecular volume to the aptamer, which provided an effective means of retaining the aptamer within the nanopores of the silica material.

In order to determine whether the tripartite complex remained intact and active upon entrapment in sol-gel derived media, emission spectra were obtained from streptavidin-bound aptamers entrapped into bulk sol-gel monoliths prepared from either DGS or sodium silicate and compared to equimolar solutions of the intact aptamer in buffer and to the intensity of entrapped FDNA alone. In both DGS and SS glasses, the entrapped tripartite complex had an emission intensity that was similar to the complex in solution (within 10%), and more than 8-fold less intense than that of the entrapped FDNA. Given that the overall fluorescence enhancement of the tripartite complex is between 8 and 10-fold upon removal of the QDNA, these results indicate that the majority of the tripartite complex remains intact upon entrapment.

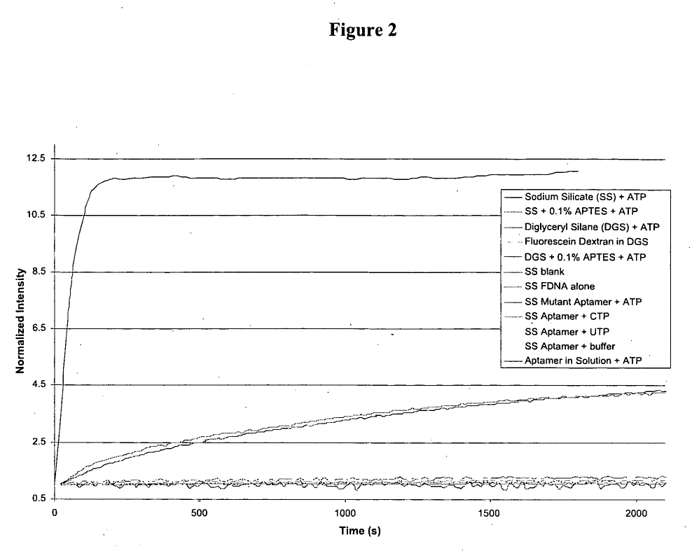

To examine the accessibility, structural-switching behaviour and fluorescence signaling capability of the entrapped aptamer, the changes in fluorescence emission intensity were examined for streptavidin-bound tripartite aptamers when entrapped into sodium silicate, SS+0.1% APTES, DGS and DGS+0.1% APTES. The following controls were included to ensure that the ATP aptamer remained both active and selective in its ATP binding capability: a blank SS sample (no sample entrapped) was used as a negative control, fluorescein dextran in DGS and FDNA alone in SS acted as positive controls to allow evaluation of maximum emission intensity values, a mutant version of the ATP aptamer which had severely reduced activity in the presence of ATP was used as another negative control, and the addition of CTP, UTP and buffer to the aptamer were used as selectivity controls. The results in FIG. 2 indicate that the ATP aptamer could selectively bind ATP and was able to undergo a structural switch to produce similar increases in fluorescence intensity after entrapment in both SS and DGS, but lost essentially all fluorescence signaling ability when APTES was used as part of the sol-gel matrix. Given that FDNA and FD were highly fluorescent in APTES doped glasses, the loss of signaling ability is not likely to be due to quenching of fluorescein by APTES, or due to a pH fluctuation since pH was held constant. In addition, the aptamer did not show signal enhancement even at very high levels of ATP (5 mM), indicating that accessibility to analyte was not likely to be the main factor contributing to inactivity of the aptamer. The loss of fluorescence signaling ability is therefore likely to be due to the DNA backbone electrostatically binding to the APTES coated silica, resulting in an inability to undergo a structural switch upon introduction of ATP.

A point that should be noted from the response of the entrapped aptamer is that the rate of the fluorescence response to the presence of ATP was much slower than was observed in solution, consistent with much slower diffusion of the ATP through the mesoporous silica network owing to both mass transport limitations and exclusion of the ATP from the anionic interior of the silica.⁵⁰ It is also noteworthy that the maximum signal enhancement in the sol-gel matrix is ˜8-fold, while in solution a maximum signal enhancement of ˜12-fold is observed. Given that the maximum signal for entrapped FDNA was similar to that in solution, it can be assumed that the entrapment has no effect on the quantum yield of FDNA. These results suggest that a fraction of the entrapped aptamer is either inactivated upon entrapment or is inaccessible to the substrate. Inaccessibility of entrapped biomolecules to anionic analytes has been reported previously, supporting the latter possibility.⁵²

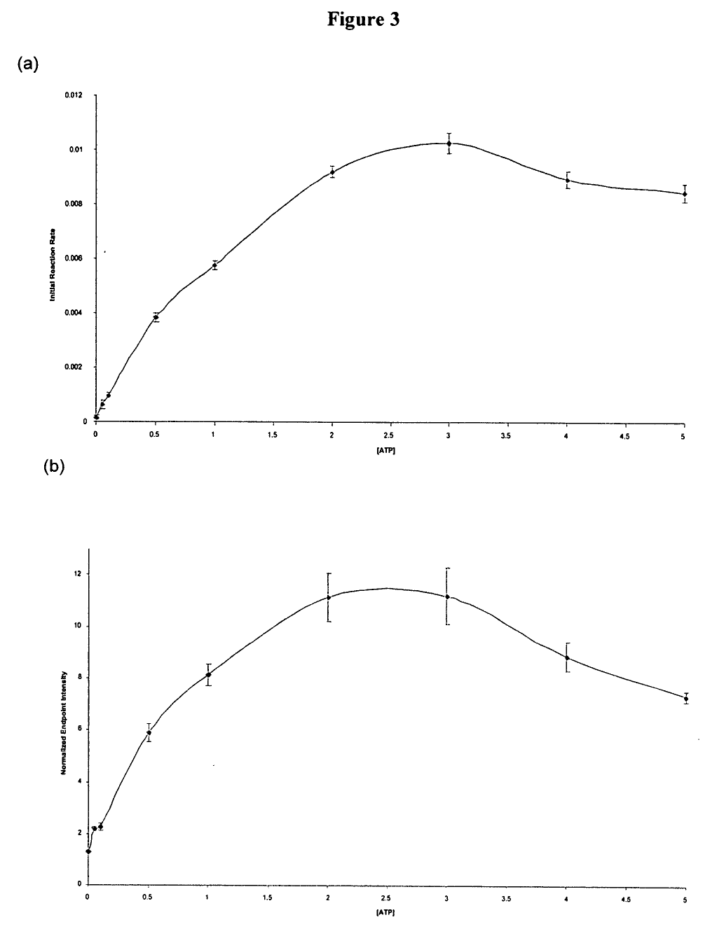

FIG. 3 shows the fluorescence response of the entrapped aptamer upon addition of various concentrations of ATP in the external solution. Panel A shows the changes in the initial slope of the response vs. ATP concentration, while Panel B shows the concentration dependent changes in the final fluorescence intensity after a 5 hour incubation with varying levels of ATP. The data show that the slope of the fluorescence response changes asymptotically with ATP concentration, as would be expected for a normal ligand binding response. The endpoint fluorescence initially increases with ATP concentration but then decreases at higher ATP concentrations. This response is similar to that observed in solution, and is due to inhibition of the aptamer by ATP at concentrations above 1 mM. Thus, use of dynamic response data (i.e., initial rates) may be useful for providing a broader dynamic range for ATP sensing. Based on the data in FIG. 3, a detection limit of 0.02 mM (3σ) and a dynamic range of 2.0 mM for the detection of ATP is estimated. Overall, the ability of the aptamer to retain activity, selectivity and signaling sensitivity upon entrapment in bulk sodium silicate indicated that the extension to a sol-gel-based microarray is possible.

Structure-Switching Aptamer Microarrays

Microarrays were produced by either pin-printing aptamer-doped sol-gel precursor solutions onto GAPS slides or by direct printing of biotinylated aptamers in a 40% glycerol solution onto neutravidin coated slide. Prior to exposure to analyte the microarray slides were allowed to age in air overnight. The arrays were formatted to include the sample of interest along with the necessary controls to allow internal signal referencing for ease of comparison of signals between arrays. All arrays included four replicates of the active tripartite aptamer complex, a mutant version of the aptamer to act as a selectivity control, a positive fluorescent control (FDNA or fluorescein dextran—70,000 MW), and a blank spot (buffer only). This provided all the information required to accurately deduce ATP sensing activity in a single experiment with replicates.

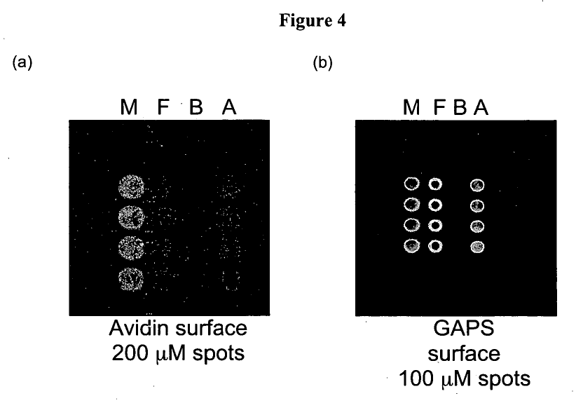

FIG. 4 illustrates the difference in sample intensity between the neutravidin coated slide (Panel A) and the sol-gel derived microarray (Panel B) before addition of ATP. The integrated intensity of the microarray spots was determined and compared to the background intensity to evaluate signal (S) to background (B) ratios. The sol-gel derived samples had a S/B ratio of approximately 250:1, while the avidin slide had only a 2:1 S/B ratio for array elements containing the active aptamer. It should be noted that the aptamer samples printed onto the avidin coated slide were at twice the initial concentration of the sol-gel printed samples. It has been previously demonstrated that sol-gel microspots have a three-dimensional hemi-spherical shape and that they can immobilize more than 100 times more sample,³⁴ thus enhancing the signal to background ratio and the relative sensitivity over monolayer immobilization techniques. The “halo” effect for the positive control (F) on the sol-gel array has been observed previously,³³ and is thought to arise from a lensing effect wherein the dome shaped sol-gel array element directs light originating from the center of the spot away from the microscope objective. It is not clear why at higher intensities (as is the case for the mutant and aptamer spots) the intensity is more uniform across the spot.

Addition of 1 mM ATP to both the directly printed and sol-gel based arrays demonstrated that the aptamer within the sol-gel array was active, while on the directly printed microarray no changes in emission intensity were observed, even at ATP levels up to 5 mM and with incubation times of up to 48 hours. The reason for the lack of a change in intensity for the directly printed aptamer is not clear. It is possible that the QDNA was displaced from the aptamer upon deposition, and thus no changes in intensity could be obtained upon binding ATP. Alternatively, the aptamer may not be able to undergo a structural switch owing to poor accessibility to the binding site on the surface, or due to steric constraints.

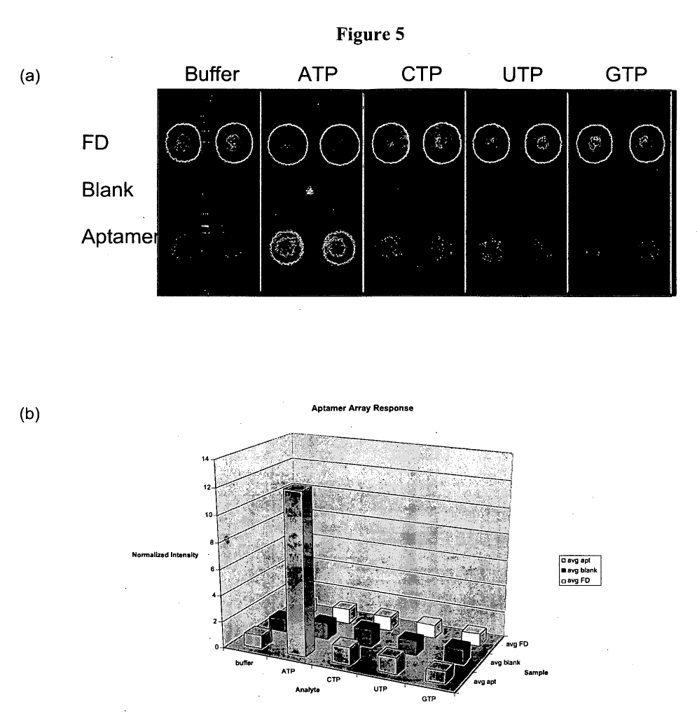

FIG. 5 illustrates the images obtained (panel A) and the relative changes in fluorescence (Panel B) observed after incubation of a sol-gel based arrays in buffer or 1 mM solutions of ATP, CTP, UTP or GTP. In all cases the intensity of the blank samples was subtracted from the fluorescent samples, and the fluorescence (positive) control was used to normalize the images for any experimental drift in laser intensity or detector sensitivity. The normalized intensity of the aptamer-loaded array elements after exposure to ATP was divided by the normalized intensity of same array elements before ATP exposure, providing a simple method to assess the relative intensity change resulting from the structure-switching process. The results clearly show that full signal enhancement occurs within the array (10-fold maximum signal change), and that the activity and selectivity of the immobilized ATP aptamer are retained in a sodium silicate derived microarray.

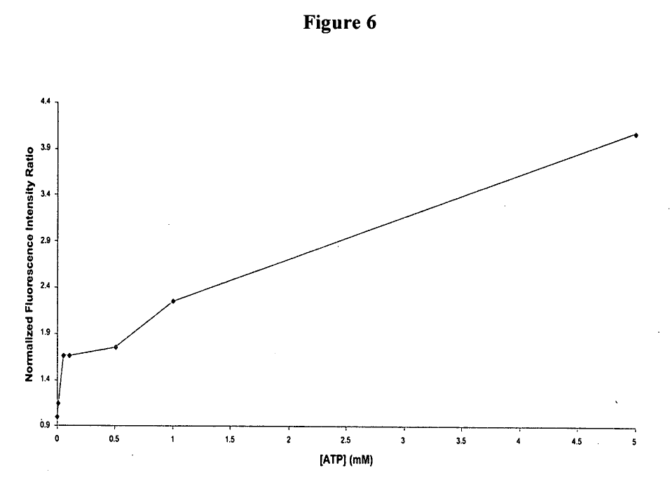

FIG. 6 shows the intensity response of the aptamer within the sol-gel based microarray to varying concentrations of ATP. Since the confocal array reader could not be configured to record time-dependent intensity values, the arrays were imaged after incubation for 4 hours with varying concentrations of ATP, and the intensity of the aptamer was determined relative to the intensity of the positive control to normalize the intensity value, and was then divided by the initial normalized intensity before addition of ATP. FIG. 6 shows that the response of the ATP aptamer within the sol-gel array is somewhat different than was obtained in bulk glasses. On the microarray the overall intensity response is somewhat lower while the dynamic range is larger than was obtained in bulk glasses. However, the detection limit is still similar to that in bulk glasses (0.02 mM), while the dynamic range is approximately 5 mM. The broader dynamic range is likely related to the relatively short incubation time (4 hr), which was chosen to avoid the potential for inhibition of aptamer at higher ATP concentrations.

### Example 2

### Study on the Entrapment of Two Forms of Structure-Switching DNA Aptamer into Biocompatible Sol-Gel Derived Materials: Tripartite Construct vs. Bipartite Construct

Methods and Materials

Chemicals. Standard oligonucleotides were prepared by automated DNA synthesis using cyanoethylphosphoramidite chemistry (Keck Biotechnology Resource Laboratory, Yale University; Central Facility, McMaster University), and purified by reversed-phase HPLC as described elsewhere.^{21,28} Tetraethylorthosilicate (TEOS), Dowex 50×8-100 cation exchange resin, Tris buffer and adenosine triphosphate, trisodium salt (ATP) were obtained from Sigma (Oakville, ON). Sodium silicate (SS, technical grade, 9% Na₂O, 29% silica, 62% water) and aminopropyltriethoxysilane (APTES) were purchased from Fisher Scientific (Pittsburgh, Pa.). Diglycerylsilane (DGS) was prepared from TEOS as described elsewhere.⁵³ Water was purified with a Milli-Q Synthesis A10 water purification system. All other chemicals and solvents used were of analytical grade.

Procedures

Preparation of DNA Aptamers: Both bipartite and tripartite structure switching aptamers were prepared, with identical aptamer sequences but different primer regions for the two constructs. The tripartite aptamer has both QDNA and FDNA binding sites, while the bipartite aptamer construct contains a covalently bound fluorescein label and thus no FDNA binding region.

The specific DNA sequences used in this work were as follows:

Tripartite Aptamer:

| | |
|---|---|
| 5′-Biotin-CCTGCCACGCTCCGCTCACTGACCTGGGGGAGTATTGCGGAGGAAGGT-3′ | [SEQ ID NO:1] |

Tripartite Mutant:

| | |
|---|---|
| 5′-Biotin-CCTGCCACGCTCCGCTCACTGACCTGGGGGAGTAATGCGGAGCAAGGT-3′ | [SEQ ID NO:2] |
| FDNA: 5′-Fluorescein-GCGGAGCGTGGCAGG-3′ | [SEQ ID NO:3] |

Bipartite Aptamer: 5′-Biotin-TTTTTTTTTTFTCACTGACCTGGGGGAGTATTGCGGAGGAAGGT [SEQ ID NO: 5]

Bipartite Mutant: 5′-Biotin-TTTTTTTTTTFTCACTGACCTGGGGTAGTATTGCGGATGAAGGT [SEQ ID NO: 6]

| | | |
|---|---|---|
| Q10DNA: | 5′-CAGGTCAGTG-Dabcyl-3′ | [SEQ ID NO:7] |
| Q11DNA: | 5′-CCAGGTCAGTG-Dabcyl-3′ | [SEQ ID NO:8] |
| Q12DNA: | 5′-CCCAGGTCAGTG-Dabcyl-3′ | [SEQ ID NO:9] |
| Q13DNA: | 5′-CCCCAGGTCAGTG-Dabcyl-3′ | [SEQ ID NO:10] |
| Q15DNA: | 5′-TCCCCAGGTCAGTG-Dabcyl-3′ | [SEQ ID NO:11] |

The lighter colored nucleotides refer to the FDNA binding region, the italicized nucleotides designate the QDNA binding region, F is the site of the fluorescein label (bound to a dT nucleotide) Q is the site of a dabcyl label (bound to a dT nucleotide) and the mutations present in the mutant DNA are underlined. Note that Q12DNA was used for all studies, unless otherwise indicated.

Preparation of DNA Aptamer Complexes: Stock solutions of the various DNA components (aptamer, FDNA, QDNA) were prepared at a concentration of 10 μM in water. The tripartite complex was prepared by combining 34 μL of either the aptamer or mutant with 17 μL of FDNA and 51 μL of QDNA and then adding 102 μL of 2× assay buffer to provide a final buffer composition of 20 mM Tris buffer at pH 8.3 containing 100 mM NaCl and 5 mM MgCl₂. This 1:2:3 ratio of FDNA:aptamer:QDNA ensured that the majority of the FDNA would be annealed to the aptamer while the excess QDNA ensured proper quenching and low background fluorescence. In the bipartite scheme, the fluorescently labeled aptamer strand was combined with QDNA in a 1:3 ratio in an identical buffer system, to ensure that the fluorophore:quencher ratio was the same as in the tripartite system. In some cases, samples that were used for entrapment studies had an additional 50 μL of streptavidin (1 mg ml⁻¹) added to them to produce an aptamer:streptavidin complex in an effort to minimize leaching of the aptamer from the sol-gel matrix.

Entrapment of DNA Aptamers: Precursor sols were prepared using either sodium silicate or DGS. Sodium silicate sols (SS) was prepared by diluting 2.9 g of sodium silicate in 10 mL of ddH₂O and immediately adding 5 g of the Dowex resin. The mixture was stirred for 30 seconds and then vacuum filtered through a Buckner funnel. The filtrate was then further filtered through a 0.45 μM membrane syringe filter to remove any particulates in the solution. DGS sols were prepared by dissolving 0.5 g of freshly prepared solid DGS into 1 mL of ddH₂O. The solid dissolved in ˜5 min under gentle agitation and was used immediately after dissolution was complete. In some cases, the SS or DGS sols also contained 0.1% (v/v) of APTES to provide cationic sites within the silica matrix. In all cases, the precursor sol solution and the buffered DNA sample solution were combined in a 1:1 (v/v) ratio to provide a final volume of 40 μL of material in the well of a 96-well plate for study. Final reagent concentrations were as follows: 1.1 μM aptamer, 0.55 μM FDNA, 1.65 μM QDNA and 2.25 μM streptavidin for the tripartite system, and 0.55 μM aptamer, 1.65 μM QDNA and 2.25 μM streptavidin for the bipartite system. Gelation typically occurred over a period of 5-20 min, depending on sol composition, and the resulting gels were aged in air for 1 h. Following aging, the samples were incubated with 100 μL of buffer for at least 1 hour prior to testing.

ATP Binding Assays: Binding assays were performed for both free and entrapped aptamers. Solution assays were performed either in 96 well plates using a TECAN Safire (80 μL total volume), or in cuvettes using a Cary Eclipse Spectrofluorimeter (400 μL total volume). Assays of entrapped aptamers were done in 96 well plates by replacing the incubation buffer with a 100 μL of buffer containing varying concentrations of ATP or other nucleoside triphosphates. All ATP binding assays were performed in fluorescence mode, and used the bottom read format in cases where microplates were utilized. In all cases fluorescein was excited at 475 nm and the emission intensity was recorded at 525 nm over a period of at least 40 minutes using a time increment of 10-20 s per point. Solution intensity data were corrected for dilution factors while intensity data for entrapped aptamers was not.

Leaching of Aptamers: Leaching of entrapped aptamers was evaluated using samples that were present in 96-well plates. The aptamer was entrapped and aged in buffer for 1 h, as described above. The total fluorescence emission of the sample was initially measured, followed by removal of the supernatant and measurement of the intensity of both the supernatant and the remaining silica gel. The relative intensity of the gel and supernatant samples were compared to the total intensity to deduce the amount of aptamer that had leached from the entrapped samples.

Accessibility of Entrapped Aptamers: Iodide quenching studies were done for the bipartite aptamer in solution and in SS derived monoliths in the absence of QDNA. For solution studies, a 0.5 μM aptamer solution was titrated by adding varying aliquots of 6.0 M potassium iodide in buffer, with spectra obtained over the full emission range of the fluorescein. Spectra were corrected for sample dilution and were integrated to obtain fluorescence intensity values. For studies of the entrapped aptamer, individual 40 μL monolithic samples in 96-well plates containing 0.5 μM bipartite aptamer, were formed at the same time and aged identically prior to incubation with varying concentrations of potassium iodide for 20 min, 60 min or 24 h. A fluorescence spectrum was collected from each sample and integrated to obtain fluorescence intensity values. Data from the monoliths incubated with different quencher concentrations were combined to yield quenching curves. All quenching data were analyzed using the following equation:

$$\frac{{F}_{0}}{Δ F} = \frac{1}{{f}_{a} {K}_{a} [ Q ]} + \frac{1}{{f}_{a}}$$

where F₀ is the fluorescence intensity in the absence of the quencher, ΔF is the difference in fluorescence intensity at a given molar concentration of quencher [Q] relative to F₀, f_{α} is the fraction of accessible fluorophore and K_{α} is the Stern-Volmer quenching constant (M⁻¹) for the accessible fluorophore. A plot of F₀/ΔF vs. 1/[Q] yields 1/f_{α} as the y-intercept and 1/f_{α}K_{α} as the slope.

Effects of QDNA Strand Length: The efficiency of the structure-switching process depends on the ability of the aptamer to displace the QDNA upon binding ATP.²⁹ To determine the effect of QDNA strand-length on the performance of entrapped aptamers, the bipartite construct was hybridized with five different QDNA strands with hybridization regions ranging from 10-15 nucleotides. The bipartite aptamer was chosen for this study to reduce fluctuations in intensity that could arise from dehybridizaton of the FDNA strand in the tripartite construct. The QDNA strands were combined with the F-aptamer at a molar ratio of 3:1 in 96 well plates. The complexes were entrapped in sodium silicate gels at a final concentration of 85 nM of aptamer. Upon aging as described above, the samples were equilibrated with 40 μL of buffer for 1 hour, followed by removal of the buffer and addition of 40 μL of 0.5 mM ATP. Both the temporal evolution of the signal and the absolute and relative changes in signal intensity were monitored.

Signal Enhancement of Bipartite vs. Tripartite Aptamer

To ensure that the bipartite system worked as well as the tripartite complex in solution, its signal enhancement upon ATP binding was tested. In comparison to the tripartite complex, the response rate of the bipartite system in solution was very similar, however, the extent of signal enhancement was not as large (8.5-fold for bipartite, 12-fold for tripartite, see below). This is likely owing to a slightly larger distance between the fluorescein and Dabcyl quencher in the case of the bipartite construct. Due to the sensitive Förster distance dependence involved in energy transfer systems, it is likely that this small difference is accountable for the slightly higher background intensity and thus smaller signal enhancement. The increase observed for the bipartite system is, however, still quite significant. In both the tripartite and bipartite complexes, the addition of streptavidin (SA) to the solution to form an aptamer-SA complex did not alter the rate of signal generation, but did result in a minor (10%) drop in final intensity. The bipartite system was advantageous for sol-gel entrapment study because it removes the possibility of false signaling that could occur due to FDNA dehybridization during entrapment.

Factors Influencing Entrapped Aptamer Activity

For proper performance of the entrapped aptamer it is optimal that: 1) the fluorophore-labeled aptamer remains fully hybridized with QDNA upon entrapment; 2) the aptamer does not leach from the matrix; and 3) the aptamer be accessible to externally added ATP. In addition, the aptamer should retain sufficient conformational flexibility to be able to undergo the structural switch upon binding ATP, and the QDNA should be able to move sufficiently far from the aptamer to produce a large fluorescence intensity increase. Each of these issues is discussed in more detail below.

(a) Aptamer-QDNA Hybridization:

Hybridization was examined only for the tripartite system, since this allows for examination of both FDNA and QDNA. We expect that QDNA hybridization should be identical in both tripartite and bipartite systems. In order to determine whether the tripartite complex remained intact and active upon entrapment in sol-gel derived silica, emission spectra were obtained from streptavidin-bound aptamers entrapped into bulk sol-gel monoliths prepared from either DGS or SS, and compared to equimolar solutions of the intact aptamer and to the intensity of entrapped FDNA alone. DGS and SS were chosen due to their proven compatibility with numerous biomolecules, many of which demonstrate diminished activity in alkoxysilane-derived silicates.^{50,54}

As shown in Table 1, the intensity of fluorescence for FDNA alone or in the presence of a 3-fold molar excess of QDNA are essentially identical in solution, while the FDNA bound to the aptamer undergoes an increase in fluorescence intensity of nearly 2.5-fold relative to FDNA alone. The FDNA-aptamer-QDNA system shows the expected decrease in emission intensity (ca. 12-fold lower than FDNA-aptamer complex). When entrapped in either DGS and SS glasses, the intensity of FDNA and FDNA-aptamer systems are essentially identical to the values obtained in solution, as are the intensity values of the FDNA/QDNA mixture, showing that the FDNA and QDNA do not interact directly even in the entrapped state. The emission intensity of the entrapped tripartite complex, which was preassembled in solution prior to entrapment, showed a significant decrease in intensity relative to the entrapped FDNA-aptamer system, however, the signal of the entrapped tripartite aptamer was slightly higher than in solution, leading to an overall reduction in the relative signal change for the entrapped aptamer (10-fold vs. 12-fold). Addition of excess QDNA (to reach a 5-fold molar excess) did not result in further quenching of the signal. The results are consistent with dehybridization of either FDNA or QDNA from only a small fraction of the entrapped aptamers.

(b) Leaching:

Leaching experiments were performed for both the tripartite and bipartite aptamer complexes in the absence of streptavidin and after formation of the aptamer-streptavidin complex in each of four sol-gel derived samples: DGS, DGS with 0.1% APTES, SS and SS with 0.1% APTES (Table 2). APTES was added to promote electrostatic retention of the DNA, while streptavidin was added to provide a larger molecular volume to the aptamer, which would be expected to aid in retention of the aptamer within the pores of the material. In general, the bipartite aptamer showed less leaching than the tripartite aptamer for a given silica material, although the trends in leaching as a function of material composition were similar. Specific values of leaching for all samples tested are listed in Table 2.

Leaching followed the trend of DGS>SS>DGS/APTES>SS/APTES, with the exception of the tripartite aptamer, which had similar leaching in both of the APTES doped materials. Intriguingly, the addition of streptavidin to either the bipartite or tripartite aptamers had no effect on leaching, within error. While this result is unexpected, it may be partially due to the use of small silica discs, which would have a much higher surface-area-to-volume ratio than larger monoliths used in previous studies, resulting in a higher proportion of easily leached surface-bound biomolecules. The result also suggests that DNA itself is sufficiently large to resist leaching from the internal pores in the bulk of the glass.

The largest extent of leaching was observed for the tripartite aptamer in DGS (49%), while the lowest amount was for the bipartite aptamer in SS/APTES (12%). The larger degree of leaching for tripartite constructs relative to bipartite constructs is most likely due to loss of the FDNA fragment, which is only 15 nucleotides long and can easily move in and out of the porous material if not properly hybridized to the aptamer primer. The FDNA cannot be distinguished from the FDNA-aptamer complex using fluorescence intensity measurements. While not wishing to be limited by theory, the reduction in leaching in SS relative to DGS is likely related to the more open pore structure in DGS materials.⁵³ The overall degree of leaching is relatively high compared to proteins,⁵⁵ and is likely a result of the electrostatic repulsion of DNA from the silica surface, which likely prevents silica templating around the DNA, and thus results in relatively high mobility for the entrapped biomolecule. The decrease in leaching upon addition of APTES to the silica is due to the fact that DNA is anionic while APTES is cationic. However, the inability to completely eliminate leaching suggests that not all DNA molecules are strongly associated with aminosilane moieties.

(c) Accessibility:

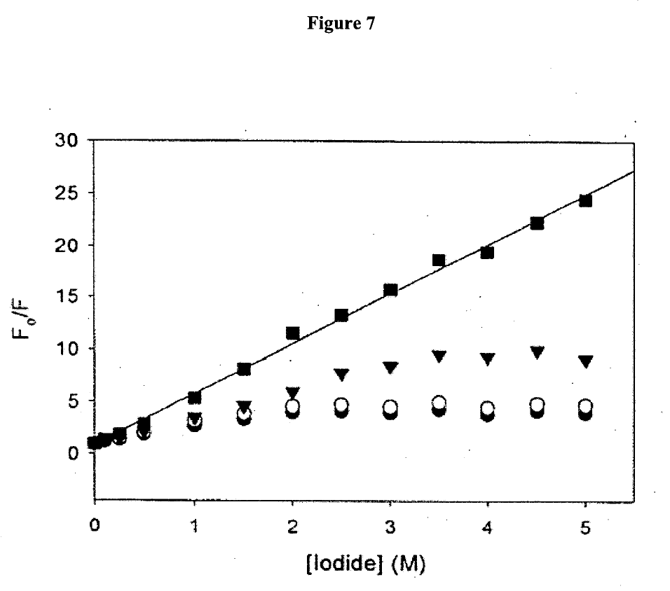

Given that ATP is a trianion, and thus may be electrostatically repelled from the silica matrix, the accessibility of the aptamer to the anionic quencher iodide was also investigated. In this case, the bipartite aptamer was used to avoid any issues with free FDNA in the matrix. FIG. 7 shows the Stern Volmer (SV) plots for free and entrapped aptamers. The solution based aptamer sample has a linear SV plot, indicative of full accessibility to iodide; however the curves of the entrapped samples start off linear but then turn sharply downward to become parallel with the x-axis. The initial linear portion of the entrapped sample curve extends to higher concentrations upon prolonged exposure to the quencher. The data point to a temporal dependence of aptamer accessibility to the anionic analyte. Fractional accessibility was found to increase from ˜90% at 20 or 60 min to 97% at 24 hrs. Hence, we would expect that up to 90% of the signal obtained in solution should be retrieved from entrapped aptamer when using incubation times in the 20-60 min range, although the larger size and higher charge of ATP relative to iodide may result in increased exclusion of analyte from the aptamer. This point is discussed further below.

(d) Signal Generation from the Entrapped Aptamer:

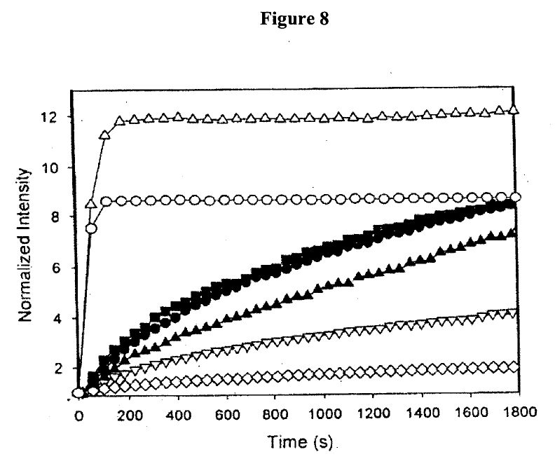

The structural-switching behavior and fluorescence signaling capability of the entrapped aptamer were examined for streptavidin-bound tripartite aptamers when entrapped in SS, SS+0.1% APTES, DGS and DGS+0.1% APTES. The tripartite construct was chosen due to its higher overall signaling capability in solution, as shown in FIG. 8. Samples were copiously washed prior to addition of ATP to remove surface-bound aptamer, and thus reduce false signals due to leaching. In this case, the added APTES was used not only to prevent leaching of aptamer, but also to minimize the potential for electrostatic exclusion of the anionic analyte ATP from the matrix.

The results in FIG. 8 indicate that the aptamer could bind ATP (present at a concentration of 2 mM to generate the maximum attainable signal) and was able to undergo a structural switch to produce an increase in fluorescence intensity. In all cases, the rate of response for the entrapped aptamer was significantly slower than in solution, consistent with much slower diffusion of the ATP through the mesoporous silica network owing to both mass transport limitations and exclusion of the ATP from the anionic interior of the silica.⁵⁰ Mass transport limitations can likely be minimized by reducing the volume of the sol-gel material through which the analyte must travel prior to reaching the target by, for example, employing thin silica films.⁵⁶

Although the rate of signal evolution was slower for entrapped aptamers, similar increases in fluorescence intensity were obtained relative to solution after entrapment in both SS and DGS. However, the aptamer experienced reduced fluorescence signaling ability when APTES was used as part of the sol-gel matrix. Given that FDNA was highly fluorescent in APTES-doped glasses, the loss of signaling ability is not likely to be due to quenching of fluorescein by APTES, or due to a pH fluctuation, since pH was held constant. While not wishing to be limited by theory, the lowered fluorescence signaling ability is therefore attributed to the DNA backbone of the aptamer electrostatically binding to the APTES coated silica, resulting in an inability to undergo a structural switch upon introduction of ATP.

The higher activity of the tripartite aptamer in SS+0.1% APTES relative to DGS+0.1% APTES is postulated to occur due to the presence of glycerol in DGS, which is a byproduct of hydrolysis and condensation. It has been reported that glycerol can have a destabilizing effect on double stranded DNA, effectively reducing the melting temperatures by as much as 20° C.⁵⁷ It has been suggested that glycerol interacts with the polynucleotide solvation sites by replacing water and by modifying electrostatic interactions between polynucleotides and their surrounding atmosphere of counterions.⁵⁸ Considering that the melting temperatures (Tₘ) of these short synthetic strands is approximately 40° C.,²¹ any reduction in Tₘ would result in a higher proportion of unhybridized DNA when working at ambient temperatures. This, coupled with the presence of APTES, which electrostatically immobilizes the DNA backbone to the surface of the matrix, likely hinders the reformation of the tripartite complex upon melting. Thus, the disruption of the DNA complex and inhibition of structural switching should severely reduce the signal enhancement of the aptamer in DGS derived materials containing APTES. To test this combined effect, the tripartite aptamer was entrapped in SS containing 0.1% APTES and 15% v/v glycerol (the same amount of glycerol as would be present in unwashed DGS derived materials).⁵³ The signaling enhancement of the sample was reduced to the level of the DGS+0.1% APTES sample, which was approximately 25% of the enhancement seen for SS+0.1% APTES without addition of glycerol.

(e) Effect of QDNA Length:

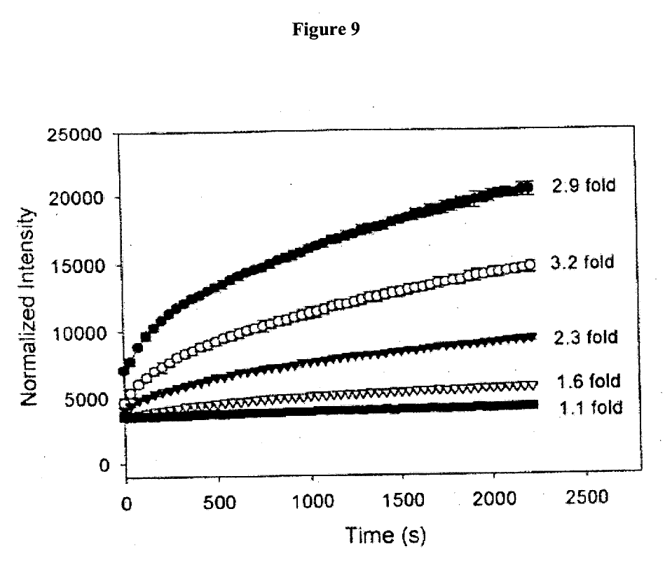

As noted above, the response of the entrapped aptamer to ATP is significantly slower than is obtained in solution. Given that the signaling ability of structure-switching aptamers can be manipulated by altering the length of the QDNA strand,²¹ we sought to examine whether such a method might also be used to alter the rate and magnitude of signal development by modifying the energy barrier necessary for QDNA removal. For this purpose, we both increased and decreased the length of the QDNA strand to create strands ranging from 10-15 nucleotides. The bipartite aptamer was used to measure the variability in response arising from the QDNA strand length modification, to assure that no additional signaling was obtained from dehybridization of FDNA. As shown in FIG. 9, upon addition of 0.5 mM ATP, the bipartite aptamer signal evolves either too slowly or not at all when too many nucleotides are added to the QDNA. On the other hand, the use of shorter QDNA strands, while imparting moderate improvements in the rate of signal evolution, also results in higher background fluorescence, consistent with poorer hybridization.

Overall, the Q11DNA provided the best compromise between signaling capability and response time for the entrapped aptamer. Previous studies have shown that the use of either the Q11DNA or Q12DNA strands provide optimal signaling performance for structure-switching aptamers in solution.²¹ In this work we chose to use the Q12DNA for most studies to allow direct comparison with solution-based studies. The difference in signaling rate and magnitude for Q11DNA and Q12DNA are not substantially different, and thus the Q12DNA provides sufficient signal enhancement and a broad dynamic range for ATP sensing within the physiological range, as shown below.

(f) Entrapped Aptamer Selectivity and Response to ATP:

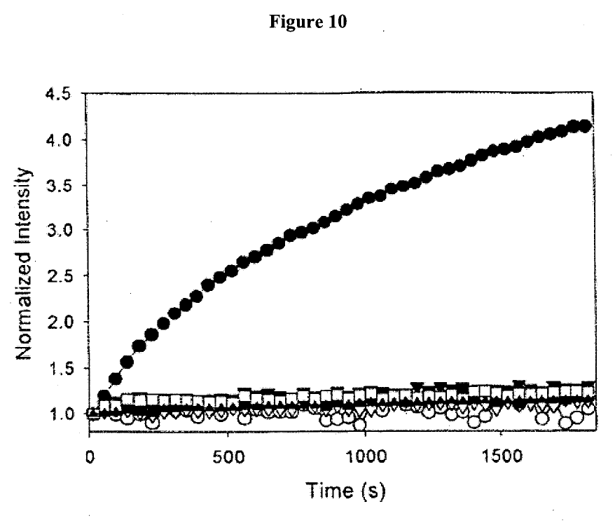

To assess the selectivity of the immobilized aptamer for ATP over other nucleotides, both native and mutant versions of the tripartite construct were immobilized in SS and examined in the presence of different nucleotides. The mutant aptamer had a change in sequence that severely reduced binding activity in the presence of ATP.²¹ The tripartite construct was used for this work owing to the higher overall signal change for this construct relative to the bipartite aptamer (FIG. 8). In addition, a blank SS sample (no aptamer entrapped) was used as a negative control, while fluorescein dextran in DGS was used as a positive control to allow for evaluation of external fluctuations in intensity values due to changes of pH or excitation intensity. As was observed in solution,²¹ the ATP aptamer responded to the addition of ATP, but not CTP, GTP or UTP, while the mutant remained quenched even in the presence of ATP (FIG. 10). The level of signal enhancement is less than that shown in FIG. 8 due to the lower level of ATP (0.5 mM) used in the selectivity study as compared to the signaling study (2 mM). Overall, the data demonstrate that the aptamer remains selective upon entrapment, and that the integrity of its binding site is not compromised.

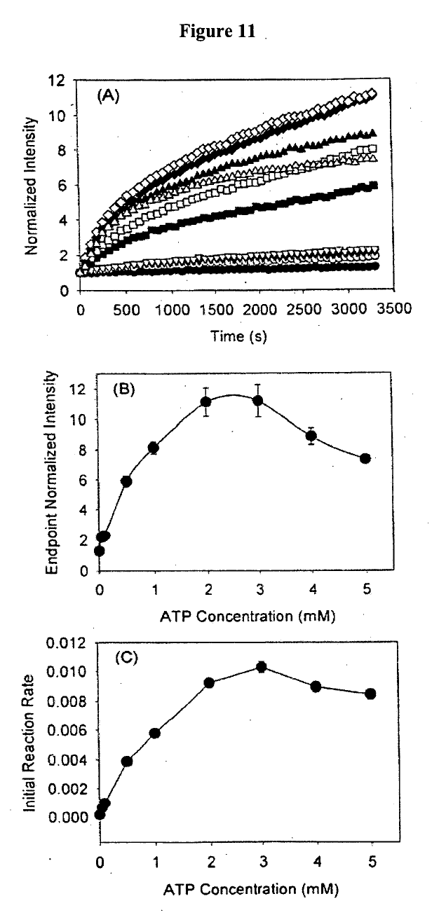

FIG. 11 shows the fluorescence response of the tripartite aptamer bearing a Q12DNA strand when entrapped in SS derived materials upon addition of various concentrations of ATP in the external solution. Panel A shows the temporal response curves for different levels of ATP. Panel B shows the concentration-dependent changes in the final fluorescence intensity after an incubation time of 1 h with varying levels of ATP, while Panel C shows the changes in the initial slope of the response vs. ATP concentration. The data show that the slope of the fluorescence response changes asymptotically with ATP concentration, as would be expected for a normal ligand-binding response. The endpoint fluorescence initially increases with ATP concentration but then reaches a plateau and decreases at higher ATP concentrations. This response is similar to that observed in solution, and is due to inhibition of the aptamer by ATP, which occurs at ATP concentrations above 10 mM in solution.¹⁷ Thus, the use of dynamic response data (i.e. initial rates) may be useful for providing a broader dynamic range for ATP sensing, and also leads to a shorter analysis time since it is not necessary to wait until a steady-state signal is reached. A point that should be noted is that the dynamic range of the entrapped aptamer matches well with the physiological range of ATP, and thus could potentially be used for direct sensing of ATP without the need for sample dilution. Furthermore, the entrapment of the DNA should minimize digestion of the DNA by nucleases present in the biological system, as has been observed for entrapped proteins.⁵⁹

While the present invention has been described with reference to the above examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
Comparison of intensity values for free and entrapped FDNA,
FDNA/QDNA, FDNA-aptamer and FDNA-aptamer-QDNA. All intensity
data is normalized to the intensity of FDNA-aptamer.

| Sample | Solution | SS | DGS |
|---|---|---|---|
| FDNA | 0.43 ± 0.01 | 0.63 ± 0.01 | 0.63 ± 0.02 |
| FDNA/QDNA | 0.40 ± 0.03 | 0.55 ± 0.02 | 0.56 ± 0.02 |
| FDNA-aptamer | 1 | 1 | 1 |
| FDNA-aptamer-QDNA | 0.08 ± 0.01 | 0.10 ± 0.01 | 0.10 ± 0.01 |

TABLE 2
Leaching Data for aptamers in different silica materials,
reported as % of total aptamer leached from the material.

| | | SS + 0.1% | | DGS + 0.1% |
|---|---|---|---|---|
| Sample | SS | APTES | DGS | APTES |
| Bipartite Aptamer | 22 ± 1 | 12 ± 1 | 38 ± 2 | 29 ± 2 |
| Bipartite Aptamer + | 18 ± 1 | 18 ± 2 | 43 ± 1 | 30 ± 2 |
| Avidin | | | | |
| Tripartite Aptamer | 43 ± 2 | 27 ± 2 | 49 ± 1 | 24 ± 1 |
| Tripartite Aptamer + | 41 ± 2 | 26 ± 2 | 46 ± 1 | 25 ± 2 |
| Avidin | | | | |

### FULL CITATIONS FOR DOCUMENTS REFERRED TO IN THE SPECIFICATION

1. Ellington, A. D.; Szostak, J. W. In vitro selection of RNA molecules that bind specific ligands. Nature 1990, 346, 818-822.
2. Tuerk, C.; Gold, L. Systematic evolution of ligands by exponential enrichment: RNA ligands to bacteriophage T4 DNA polymerase. Science 1990, 249, 505.
3. Famulok, M.; Mayer, G.; Blind, M. Nucleic acid aptamers—from selection in vitro to applications in vivo. Acc. Chem. Res. 2001, 33, 591.
4. Wilson, D. S.; Szostak, J. W. In vitro selection of functional nucleic acids. Annu. Rev. Biochem. 1999, 68, 611.
5. Green, L. S.; Jellinek, D.; Bell, C.; Beebe, L. A.; Feistner, B. D.; Gill, S. C.; Jucker, F. M.; Janjic, N. Nuclease-resistant nucleic acid ligands to vascular permeability factor/vascular endothelial growth factor. Chem. Biol. 1995, 2, 683.
6. Pagratis, N. C.; Bell, C.; Chang, Y. F.; Jennings, S.; Fitzwater, T.; Jellinek, D.; Dang, C. Potent 2′-amino-, and 2′-fluoro-2′-deoxyribonucleotide RNA inhibitors of keratinocyte growth factor. Nat. Biotechnol. 1997, 15, 68.
7. Green, L. S.; Jellinek, D.; Jenison, R.; Ostman, A.; Heldin, C. H.; Janjic, N. Inhibitory DNA ligands to platelet-derived growth factor B-chain. Biochemistry 1996, 35, 14413.
8. Jenison, R. D.; Gill, S. C.; Pardi, A.; Polisky, B. High-resolution molecular discrimination by RNA. Science 1994, 263, 1425.
9. Geiger, A.; Burgstaller, P.; von der Eltz, H.; Roeder, A.; Famulok, M. RNA aptamers that bind L-arginine with sub-micromolar dissociation constants and high enantioselectivity. Nucleic Acids Res. 1996, 24, 1029.
10. Jhaveri, S.; Kirby, R.; Conrad, R.; Maglott, E.; Bowser, M.; Kennedy, R. T.; Glick, G.; Ellington, A. D. Designed Signaling Aptamers that Transduce Molecular Recognition to Changes in Fluorescence Intensity. J. Am. Chem. Soc. 2000, 122, 2469.
11. Jhaveri, S.; Rajendran, M.; Ellington, A. D. In vitro selection of signaling aptamers. Nat. Biotechnol. 2000, 18, 1293.
12. Hamaguchi, N.; Ellington, A.; Stanton, M. Aptamer beacons for the direct detection of proteins. Anal. Biochem. 2001, 294, 126.
13. Yamamoto, R.; Baba, T.; Kumar, P. K. Molecular beacon aptamer fluoresces in the presence of Tat protein of HIV-1. Genes Cells 2001, 5, 389.
14. Li, J. J.; Fang, X.; Tan, W. Molecular aptamer beacons for real-time protein recognition. Biochem. Biophys. Res. Commun. 2002, 292, 31.
15. Stojanovic, M. N.; de Prada, P.; Landry, D. W. Aptamer-based folding fluorescent sensor for cocaine. J. Am. Chem. Soc. 2001, 123, 4928.
16. Stojanovic, M. N.; de Prada, P.; Landry, D. W. Fluorescent Sensors Based on Aptamer Self-Assembly. J. Am. Chem. Soc. 2000, 122, 11547.
17. Fang, X.; Sen, A.; Vicens, M.; Tan, W. Synthetic DNA aptamers to detect protein molecular variants in a high-throughput fluorescence quenching assay. Chembiochem 2003, 4, 829.
18. Karlstrom, A.; Nygren, P. A. Dual labeling of a binding protein allows for specific fluorescence detection of native protein. Anal. Biochem. 2001, 295, 22.
19. Yamana, K.; Ohtani, Y.; Nakano, H.; Saito, I. Bis-pyrene labeled DNA aptamer as an intelligent fluorescent biosensor. Bioorg. Med. Chem. Lett. 2003, 13, 3429.
20. Merino, E. J.; Weeks, K. M. Fluorogenic resolution of ligand binding by a nucleic acid aptamer. J. Am. Chem. Soc. 2003, 125, 12370.
21. Nutiu, R.; Li, Y. Structure-switching signaling aptamers: transducing molecular recognition into fluorescence signaling. Chem. Eur. J. 2004, 10, 1868.
22. Tyagi, S.; Kramer, F. R. Molecular beacons: probes that fluoresce upon hybridization. Nat. Biotechnol. 1996, 14, 303. (this appears as 16 in text)
23. Wilson, C.; Szostak, J. W. Isolation of a fluorophore-specific DNA aptamer with weak redox activity. Chem. Biol. 1998, 5, 609.
24. Holeman, L. A.; Robinson, S. L.; Szostak, J. W.; Wilson, C. Isolation and characterization of fluorophore-binding RNA aptamers. Fold. Des. 1998, 3, 423.
25. Babendure, J. R., Adams, S. R.; Tsien, R. Y. Aptamers switch on fluorescence of triphenylmethane dyes. J. Am. Chem. Soc. 2003, 125, 14716.
26. Jiang, Y.; Fang, X.; Bai, C. Signaling aptamer/protein binding by a molecular light switch complex. Anal. Chem. 2004, 76, 5230.
27. Stojanovic, M. N.; Kolpashchikov, D. M. Modular aptameric sensors. J. Am. Chem. Soc. 2004, 126, 9266.
28. S. Mei, Z. Liu, J. D. Brennan and Y. Li*. An Efficient RNA-cleaving DNA Enzyme That Synchronizes Catalysis with Fluorescence Signalling. J. Am. Chem. Soc., 2003, 125, 412-420.
29. Nutiu, R.; Li, Y. Structure-switching signaling aptamers. J. Am. Chem. Soc. 2003, 125, 4771.
30. McCauley, T. G.; Hamaguchi, N.; Stanton, M. Aptamer-based biosensor arrays for detection and quantification of biological macromolecules. Anal. Biochem. 2003, 319, 244.
31. Lee, M.; Walt, D. R. A Fiber-Optic Microarray Biosensor Using Aptamers as Receptors. Anal. Biochem. 2000, 282, 142.
32. Potyrailo, R. A.; Conrad, R. C.; Ellington, A. D.; Hieftje, G. M. Adapting Selected Nucleic Acid Ligands (Aptamers) to Biosensors. Anal. Chem. 1998, 70, 3419.
33. Rupcich, N.; Brennan, J. D. Coupled enzyme reaction microarrays based on pin-printing of sol-gel derived biomaterials. Anal. Chim. Acta 2003, 500, 3.
34. Rupcich, N.; Goldstein, A. M.; Brennan, J. D. Optimization of Sol-Gel Formulations and Surface Treatments for the Development of Pin-Printed Protein Microarrays. Chem. Mater. 2003, 15, 1803.
35. Cho, E. J.; Bright, F. V. Optical Sensor Array and Integrated Light Source. Anal. Chem. 2001, 73, 3289.
36. Cho, E. J.; Bright, F. V. Pin-Printed Chemical Sensor Arrays for Simultaneous Multianalyte Quantification. Anal. Chem. 2002, 74, 1462.
37. Cho, E. J.; Bright, F. V. Integrated chemical sensor array platform based on a light emitting diode, xerogel-derived sensor elements, and high-speed pin printing. Anal. Chim. Acta. 2002, 470, 101.
38. Li, Jun; Tan, Weihong; Wang, Kemin; Yang, Xiaohai; Tang, Zhiwen; He, Xiaoxiao. Optical DNA biosensor based on molecular beacon immobilized on sol-gel membrane. Proc. SPIE-Int. Soc. Opt. Eng. 2001, 4414, 27-30.
39. Mandal M, Breaker R R. Adenine riboswitches and gene activation by disruption of a transcription terminator. Nat Struct Mol. Biol. 2004 January; 11(1):29-35.
40. Nahvi A, Barrick J E, Breaker R R. Coenzyme B 12 riboswitches are widespread genetic control elements in prokaryotes. Nucleic Acids Res. 2004 Jan. 2; 32(1): 143-50.
41. Sudarsan N, Wickiser J K, Nakamura S, Ebert M S, Breaker R R. An mRNA structure in bacteria that controls gene expression by binding lysine. Genes Dev. 2003 Nov. 1; 17(21):2688-97.
42. Winkler W C, Cohen-Chalamish S, Breaker R R. An mRNA structure that controls gene expression by binding FMN. Proc Natl Acad Sci USA. 2002 Dec. 10; 99(25):15908-13.
43. Winkler W C, Nahvi A, Sudarsan N, Barrick J E, Breaker R R. An mRNA structure that controls gene expression by binding S-adenosylmethionine. Nat Struct Biol. 2003 September; 10(9):701-7.
44. Winkler W, Nahvi A, Breaker R R. Thiamine derivatives bind messenger RNAs directly to regulate bacterial gene expression. Nature. 2002 Oct. 31; 419(6910):952-6.
45. Mandal M, Boese B, Barrick J E, Winkler W C, Breaker R R. Riboswitches control fundamental biochemical pathways in Bacillus subtilis and other bacteria. Cell. 2003 May 30; 113(5):577-86.
46. Sudarsan N, Barrick J E, Breaker R R. Metabolite-binding RNA domains are present in the genes of eukaryotes. RNA. 2003 June; 9(6):644-7.
47. Nahvi A, Sudarsan N, Ebert M S, Zou X, Brown K L, Breaker R R. Genetic control by a metabolite binding mRNA. Chem Biol. 2002 September; 9(9): 1043.
48. Kujau, M. J.; Wölfl, S. Intramolecular derivatization of 2′-amino-pyrimidine modified RNA with functional groups that is compatible with re-amplification. Nucleic Acids Res. 1998, 26, 1851.
49. Kubik, M. F.; Bell, C.; Fitzwater, T.; Watson, S. R.; Tasset, D. M. Isolation and characterization of 2′-fluoro-, 2′-amino-, and 2′-fluoro-/amino-modified RNA ligands to human IFN-g that inhibit receptor binding. J. Immuno. 1997, 159, 259.
50. Besanger, T. R.; Chen, Y.; Deisingh, A. K.; Hodgson, R.; Jin, W.; Mayer, S.; Brook, M. A.; Brennan, J. D. Screening of Inhibitors Using Enzymes Entrapped in Sol-Gel-Derived Materials. Anal. Chem. 2003, 75, 2382.
51. Tleugabulova, D.; Zhang, Z.; Chen, Y.; Brook, M. A.; Brennan, J. D. Fluorescence Anisotropy in Studies of Solute Interactions with Covalently Modified Colloidal Silica Nanoparticles. Langmuir, 2004, 20, 848-854.
52. Flora, K. K.; Brennan, J. D. Effect of Matrix Aging on the Behavior of Human Serum Albumin Entrapped in a Tetraethyl Orthosilicate-Derived Glass. Chem. Mater. 2001, 13, 4170.
53. a) Brook, M. A.; Chen, Y.; Guo, K.; Zhang, Z.; Jin, W.; Deisingh, A.; Brennan, J. D. Proteins Entrapped in Silica Monoliths Prepared from Glyceroxysilanes. J. Sol-Gel Sci. Technol. 2004, 31, 343-348. b) Brook, M. A.; Chen, Y.; Guo, K.; Zhang, Z.; Brennan, J. D. Sugar-modified silanes: precursors for silica monoliths. J. Mater. Chem. 2004, 14, 1469-1479.
54. Bhatia, R. B.; Brinker, C. J.; Gupta, A. K.; Singh, A. K. Aqueous Sol-Gel Process for Protein Encapsulation. Chem. Mater. 2000, 12, 2434-2441.
55. Zheng, L.; Reid, W. R.; Brennan, J. D. Measurement of Fluorescence from Tryptophan To Probe the Environment and Reaction Kinetics within Protein-Doped Sol-Gel-Derived Glass Monoliths. Anal. Chem. 1997, 69, 3940-3949.
56. Flora, K; Brennan, J. D. Comparison of formats for the development of fiber-optic biosensors utilizing sol-gel derived materials entrapping fluorescently-labelled protein. Analyst, 1999, 124, 1455-1462.
57. Bonner, G.; Klibanov, A. M. Structural stability of DNA in nonaqueous solvents. Biotechnology and Bioengineering. 2000, 68 (3), 339.
58. Del Vecchio, P.; Esposito, D.; Ricchi, L.; Barone, G. The effects of polyols on the thermal stability of calf thymus DNA. Int. J. Biological Macromolecules. 1999, 24, 361.
59. Jin, W.; Brennan, J. D. Properties and applications of proteins encapsulated within sol-gel derived materials. Anal. Chim. Acta, 2002, 461, 1. (b) Brennan, J. D. Using Intrinsic Fluorescence to Investigate Proteins Entrapped in Sol-Gel Derived Materials. Applied Spectroscopy, 1999, 53, 106A-121A.

What is claimed is:

1. A method of immobilizing nucleic acid aptamers comprising combining at least one nucleic acid aptamer with a sol-gel precursor and treating the at least one aptamer and precursor under conditions for a gel to form.

2. The method according to claim 1, wherein the nucleic acid aptamer is part of a system.

3. The method according to claim 1, wherein the aptamer is labelled with a detectable label.

4. The method according to claim 2, wherein the aptamer system comprises a structure switching DNA aptamer, aptamer beacons or an aptamers in combination with a reporter molecules.

5. The method according to claim 4, wherein the reporter molecules are antibodies.

6. The method according to claim 4, wherein the aptamer system is a structure switching aptamer system comprising:
(a) a DNA or RNA that has been modified by addition of a fluorophore (denoted FDNA or FRNA);
(b) a DNA or RNA that has been modified by addition of a molecule that quenches fluorescence of the fluorophore (denoted QDNA or QRNA); and
(c) a DNA or RNA aptamer which comprises a nucleotide sequence that specifically binds to an analyte of interest, a nucleotide sequence that binds to the FDNA or FRNA and a nucleotide sequence that binds to the QDNA or QRNA.

7. The method according to claim 6, wherein the nucleotide sequence that specifically binds to an analyte of interest is biotinylated.

8. The method according to claim 6, wherein (a), (b) and (c) are assembled into a tripartite structure.

9. The method according to claim 4, wherein the aptamer system is a structure switching aptamer system comprising
(a) a DNA or RNA that has been modified by addition of a molecule that quenches fluorescence of a fluorophore (denoted QDNA or QRNA); and
(b) a DNA or RNA aptamer which comprises a nucleotide sequence that specifically binds to an analyte of interest, a nucleotide sequence that has been modified by addition of the fluorophore and a nucleotide sequence that binds to the QDNA or QRNA.

10. The method according to claim 9, wherein the nucleotide sequence that specifically binds to an analyte of interest is biotinylated.

11. The method according to claim 9 wherein (a) and (b) are assembled into a bipartite structure.

12. The method according to claim 6, wherein the fluorophore is fluorescein.

13. The method according claim 6, wherein the analyte of interest is selected from the group consisting of metal ions, small organic molecules, biological cofactors, metabolites, proteins and nucleic acids.

14. The method according to claim 13, wherein the analyte of interest is adenosine triphosphate (ATP).

15. The method according to claim 6, wherein the QDNA or QRNA comprises from 10 to 15 nucleotides.

16. The method according to claim 15, wherein the FDNA or QRNA comprises from 11 to 12 nulceotides.

17. The method according to claim 6 wherein the aptamer system comprises [SEQ ID NO: 1], [SEQ ID NO: 3] and [SEQ ID NO: 4].

18. The method according to claim 9, wherein the aptamer system comprises [SEQ ID NO: 5] and a nucleic acid sequence selected from the group consisting of [SEQ ID NO: 7], [SEQ ID NO: 8], [SEQ ID NO: 9], [SEQ ID NO: 10] and [SEQ ID NO: 11].

19. The method according to claim 18, wherein the aptamer system comprises [SEQ ID NO: 5] and a nucleic acid sequence selected from the group consisting of [SEQ ID NO: 8] and [SEQ ID NO: 9].

20. The method according to claim 2, wherein the aptamer system comprises a signaling DNA enzyme construct comprising:
(a) an enzymatic DNA sequence; and
(b) a DNA sequence having a ribonucleotide linkage flanked by a fluorophore modified oligonucleotide and a quencher modified oligonucleotide in sufficient proximity to each other whereby, in the absence of catalysis, fluorescence from the fluorophore is quenched by the quencher.

21. The method according to claim 20, wherein the signaling DNA sequence construct further comprises an aptamer sequence conjugated to the enzymatic DNA sequence.

22. The method according to claim 1, wherein the aptamer is bound, either covalently or electrostatically or by affinity interactions, to another molecule that inhibits leaching of the aptamer or aptamer system from the sol-gel.

23. The method according to claim 22, wherein the molecule that inhibits leaching of the aptamer or aptamer system from the sol-gel is streptavidin.

24. The method according to claim 1, wherein the aptamer comprises DNA.

25. The method according to claim 1, wherein the sol gel is biomolecule compatible.

26. The method according to claim 25, wherein the sol gel precursor is sodium silicate or diglyceryl silane.

27. A method of immobilizing nucleic acid aptamers or nucleic acid aptamer systems in silica matrixes comprising:
(i) combining an aqueous solution of a sol-gel precursor with an aqueous solution of the nucleic acid aptamer or nucleic acid aptamer system;
(ii) adjusting the pH of the combination of (i) so that it is in the range of about 4-11;
(iii) shaping the combination into a desired shape;
(iv) allowing the combination to gel; and
(v) aging and partially drying the gel.

28. A sol gel comprising one or more nucleic acid aptamers or nucleic acid aptamer systems immobilized therein.

29. A sol gel prepared using the method according to claim 1.

30. A kit, microarray or a column comprising the sol gel according to claim 29.

31. A method for detecting a molecule comprising:
(a) exposing an aptamer, or aptamer system, that specifically binds to the molecule, said aptamer or aptamer system being immobilized in a sol-gel, to a test solution suspected of comprising the molecule; and
(b) detecting a change in one or more characteristics of aptamer or aptamer system.

32. The method according to claim 31, wherein the sol-gel is as defined in claim 29.

33. The method according to claim 32, wherein a change in the one or more characteristics of the aptamer or aptamer system in the presence of the molecule compared to one or more controls indicates that the molecule was present in the solution.

34. A method of separating one or more compounds from a mixture comprising:
(a) contacting the mixture with a sol gel comprising an aptamer or aptamer system immobilized therein, said aptamer or aptamer system having selective binding to the one or more compounds, under conditions for the one or more compounds to bind to the aptamer or aptamer system;
(b) treating the sol gel under conditions to remove the mixture from the sol gel; and
(c) optionally isolating the one or more compounds from the sol gel.

35. The method according to claim 34, wherein the sol-gel is as defined in claim 29.

36. A method of performing chemical reactions comprising:
(a) exposing one or more reactants to a sol gel comprising an aptamer or aptamer system immobilized therein, under conditions for the reaction to proceed to produce one or more reaction products;
(b) removing the one or more reaction products from the sol gel; and
(c) optionally isolating the one or more reaction products.

37. The method according to claim 36, wherein the sol-gel is as defined in claim 29.
